SEQUENCE LISTING (11 sequences)

SEQ ID NO: 1 - protein, 229 aa, synthetic construct
MPKKKRKVGSGEFDALDDFDLDMLGSDALDDFDLDMLGSDALDDFDLDMLGSDALDDFDLDMLGSGGGGSGGGGSGGGGSATMMLKKILKIEELDERELIDIEVSGNHLFYANDILTHNSNRGGGGSGGGGSGGGGSGTARPGERPFQCRICMRNFSRQDRLDRHTRTHTGEKPFQCRICMRNFSQKEHLAGHLRTHTGEKPFQCRICMRNFSRRDNLNRHLKTHLRGS

SEQ ID NO: 2 - protein, 239 aa, synthetic construct
MPKKKRKVGSGEFDALDDFDLDMLGSDALDDFDLDMLGSDALDDFDLDMLGSDALDDFDLDMLGSGGGGSGGGGSGGGGSATMGSMEAKTYIGKLKSRKIVSNEDTYDIQTSTHNFFANDILVHNSEIGSNRGGGGSGGGGSGGGGSGTARPGERPFQCRICMRNFSRQDRLDRHTRTHTGEKPFQCRICMRNFSQKEHLAGHLRTHTGEKPFQCRICMRNFSRRDNLNRHLKTHLRGS

SEQ ID NO: 3 - protein, 726 aa, synthetic construct
MKLLSSIEQACDICRLKKLKCSKEKPKCAKCLKNNWECRYSPKTKRSPLTRAHLTEVESRLERLEGSMLKKILKIEELDERELIDIEVSGNHLFYANDILTHNSGSGSQLFLLIFPREDLDMILKMDSLQDIKALLTGLFVQDNVNKDAVTDRLASVETDMPLTLRQHRISATSSSEESSNKGQRQLTVSGSSEASGSGRADALDDFDLDMLGSDALDDFDLDMLGSDALDDFDLDMLGSDALDDFDLDMLINSRSSGSPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLFRS

SEQ ID NO: 4 - protein, 309 aa, synthetic construct
MSRLDKSKVINSALELLNEVGIEGLTTRKLAQKLGVEQPTLYWHVKNKRALLDALAIEMLDRHHTHFCPLEGESWQDFLRNKAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGDSGGGGSMLKKILKIEELDERELIDIEVSGNHLFYANDILTHNSGSGSGRADALDDFDLDMLGSDALDDFDLDMLGSDALDDFDLDMLGSDALDDFDLDMLIN

SEQ ID NO: 5 - protein, 829 aa, synthetic construct
MSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPGSMLKKILKIEELDERELIDIEVSGNHLFYANDILTHNSGSGSAFLFGLELIICGLEKQLKCESGSPADALDDFDLDMLPADALDDFDLDMLPADALDDFDLDMLPGQPGSSEASGSGRADALDDFDLDMLGSDALDDFDLDMLGSDALDDFDLDMLGSDALDDFDLDMLINSRSSGSPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLFRS

SEQ ID NO: 6 - protein, 285 aa, synthetic construct
MSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGSGGGGSGGGGTGGGGTGTGYNEVLNVFPKSKKKSYKITLEDGKEIICSEEHLFPTQTGEMNISGGLKEGMCLYVKEGST

SEQ ID NO: 7 - protein, 579 aa, synthetic construct
MMLKKILKIEELDERELIDIEVSGNHLFYANDILTHNSGSGSSEASGSGRADALDDFDLDMLGSDALDDFDLDMLGSDALDDFDLDMLGSDALDDFDLDMLINSRSSGSPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLFRSTSK

SEQ ID NO: 8 - protein, 410 aa, synthetic construct
MGGGGSGGGGTGGGGTGTRSGYCLDLKTQVQTPQGMKEISNIQVGDLVLSNTGYNEVLNVFPKSKKKSYKITLEDGKEIICSEEHLFPTQTGEMNISGGLKEGMCLYVKEGSGSGATNFSLLKQAGDVEENPGPGSGEGRGSLLTCGDVEENPGPIDGGSGGSATMVSKGEELFTGVVPILVELDGDVNGHKFSVSGEGEGDATYGKLTLKLICTTGKLPVPWPTLVTTLGYGLQCFARYPDHMKQHDFFKSAMPEGYVQERTIFFKDDGNYKTRAEVKFDGDTLVNRIELKGIDFKEDGNILGHKLEYNYNSHNVYITADKQKNGIKANFKIRHNIEDGGVQLADHYQQNTPIGDGPVLLPDNHYLSYQSKLSKDPNEKRDHMVLLEFVTAAGITLGMDELYKGSGSGS

SEQ ID NO: 9 - protein, 340 aa, synthetic construct
MMLKKILKIEELDERELIDIEVSGNHLFYANDILTHNSGSGSGSGATNFSLLKQAGDVEENPGPGSGEGRGSLLTCGDVEENPGPIDGGSGGSATMVSKGEELFTGVVPILVELDGDVNGHKFSVSGEGEGDATYGKLTLKLICTTGKLPVPWPTLVTTLGYGLQCFARYPDHMKQHDFFKSAMPEGYVQERTIFFKDDGNYKTRAEVKFDGDTLVNRIELKGIDFKEDGNILGHKLEYNYNSHNVYITADKQKNGIKANFKIRHNIEDGGVQLADHYQQNTPIGDGPVLLPDNHYLSYQSKLSKDPNEKRDHMVLLEFVTAAGITLGMDELYKGSGSGS

SEQ ID NO: 10 - protein, 409 aa, synthetic construct
MNPCCLVGSSEIITRNYGKTTIKEVVEIFDNDKNIQVLAFNTHTDNIEWAPIKAAQLTRPNAELVELEIDTLHGVKTIRCTPDHPVYTKNRGYVRADELTDDDELVVAIGSGSGATNFSLLKQAGDVEENPGPGSGEGRGSLLTCGDVEENPGPIDGGSGGSATMVSKGEELFTGVVPILVELDGDVNGHKFSVSGEGEGDATYGKLTLKLICTTGKLPVPWPTLVTTLGYGLQCFARYPDHMKQHDFFKSAMPEGYVQERTIFFKDDGNYKTRAEVKFDGDTLVNRIELKGIDFKEDGNILGHKLEYNYNSHNVYITADKQKNGIKANFKIRHNIEDGGVQLADHYQQNTPIGDGPVLLPDNHYLSYQSKLSKDPNEKRDHMVLLEFVTAAGITLGMDELYKGSGSGS

SEQ ID NO: 11 - protein, 343 aa, synthetic construct
MEAKTYIGKLKSRKIVSNEDTYDIQTSTHNFFANDILVHNSEIGSGSGATNFSLLKQAGDVEENPGPGSGEGRGSLLTCGDVEENPGPIDGGSGGSATMVSKGEELFTGVVPILVELDGDVNGHKFSVSGEGEGDATYGKLTLKLICTTGKLPVPWPTLVTTLGYGLQCFARYPDHMKQHDFFKSAMPEGYVQERTIFFKDDGNYKTRAEVKFDGDTLVNRIELKGIDFKEDGNILGHKLEYNYNSHNVYITADKQKNGIKANFKIRHNIEDGGVQLADHYQQNTPIGDGPVLLPDNHYLSYQSKLSKDPNEKRDHMVLLEFVTAAGITLGMDELYKGSGSGS

(19) United States
(12) Patent Application Publication
(10) Pub. No.: US 2026/0028633 A1
(43) Pub. Date: Jan. 29, 2026

(54) INTEIN-BASED CONTROLLERS

(71) Applicant: ETH ZURICH, Zurich (CH)

(72) Inventors: Stanislav ANASTASSOV, Zürich (CH); Maurice Georges FILO, Zürich (CH); Ching-Hsiang CHANG, Zürich (CH); Mustafa Hani KHAMMASH, Zürich (CH)

(73) Assignee: ETH ZURICH, Zurich (CH)

(21) Appl. No.: 18/998,502

(22) PCT Filed: Jul. 26, 2023
(86) PCT No.: PCT/EP2023/070730

§ 371 Date: Jan. 26, 2025

(30) Foreign Application Priority Data
Jul. 26, 2022 (EP) 22186956.3

(51) Int. Cl. C12N 15/67; C07K 14/47

(52) U.S. Cl. CPC C12N 15/67; C07K 14/4702; C07K 2319/92

(57) ABSTRACT

The present invention relates to an expression system and a method ensuring constant-level concentration of an output. The invention also relates to a cell comprising the expression system.

This application claims the right of priority of European Patent Application EP22186956.3 filed 26 Jul. 2022, which is incorporated by reference herein.

### FIELD

The present invention relates to an expression system and a method ensuring constant-level concentration of an output. The invention also relates to a cell comprising the expression system.

### BACKGROUND

One of the essential features of living systems is their ability to maintain a robust behavior despite disturbances coming from their external uncertain and noisy environments. This feature, in pure biological terms, is referred to as homeostasis which is typically achieved via endogenous feedback regulatory mechanisms shaped by billions of years of evolution inside the cells. Pathological diseases are often linked to loss of homeostasis. The need to restore homeostasis for preventing such diseases, when endogenous regulatory mechanisms fail, was one of the main drivers of ushering a new active field of research referred to as cybergenetics—a field that brings control theory and synthetic biology together. In particular, the rational design of biomolecular feedback controllers offers promising candidates that may accompany or even replace such failed mechanisms (C. Kemmer et al., Nature biotechnology, vol. 28, no. 4, p. 355, 2010.; K. Rössger et al.,” Nature communications, vol. 4, no. 1, pp. 1-9, 2013; M. Xie et al., Science, vol. 354, no. 6317, pp. 1296-1301, 2016). A notion which is similar to homeostasis, but more stringent, is Robust Perfect Adaptation (RPA) (F. Xiao and J. C. Doyle, IEEE, 2018, pp. 4345-4352; M. H. Khammash, Cell Systems, vol. 12, no. 6, pp. 509-521, 2021) which is the biological analogue of the well-known notion of robust steady-state tracking in control theory. A controller succeeds in achieving RPA if it drives the steady state of a variable of interest to a prescribed set-point despite varying initial conditions, plant uncertainties and/or constant disturbances. Motivated by the internal model principle (B. A. Francis and W. M. Wonham, Automatica, vol. 12, no. 5, pp. 457-465, 1976.), which establishes that the designed controller must implement an integral feedback component to be able to achieve RPA, the antithetic integral controller (C. Briat et al., Cell Systems, vol. 2, no. 1, pp. 15-26, 2016) was brought forward (see FIG. 1(a)).

The task of building genetic controllers can be divided into two sub-tasks. First, one has to design the reaction network topologies that are capable of mathematically realizing integral control structures that achieve RPA, or even more advanced structures such as proportional-integral-derivative (PID) controllers that are capable of shaping the transient response and enhancing the performance while maintaining RPA. Then, one has to find the genetic parts whose dynamics are captured by the designed reaction network topologies. The inventors have proposed, analyzed and built several reaction network topologies and genetic parts that realize partial or full PID biomolecular controllers in the inventors' previously filed patent application and the inventors' published papers (M. Filo, S. Kumar, and M. Khammash, Nature Communications, vol. 13, no. 1, pp. 1-19, 2022; S. K. Aoki et al., Nature, p. 1, 2019; T. Frei et al., PNAS, 2022, 119 (24)).

Based on the above-mentioned state of the art, the objective of the present invention is to provide means and methods for an expression system capable of ensuring constant-level concentration of an output protein. This objective is attained by the subject-matter of the independent claims of the present specification, with further advantageous embodiments described in the dependent claims, examples, figures and general description of this specification.

### SUMMARY OF THE INVENTION

In the inventors' current work, the inventors use new genetic parts based on inteins to build genetic controllers capable of achieving RPA. These new genetic parts also give rise to a broad class of new reaction network topologies.

An intein is a segment of a protein that is capable of autocatalytically cutting itself from the protein and reconnecting the remaining segments, called exteins, via peptide bonds. Inteins are universal as they can be naturally found in all domains of life spanning eukaryotes, bacteria, archaea and viruses. Split-inteins—a subset class of inteins—are, as the name suggests, inteins split into two halves commonly referred to as IntN and IntC. These small protein segments are capable of heterodimerizing and performing protein splicing reactions on their own where they break and form new peptide bonds. These features can be exploited to ligate, remove or exchange amino acid sequences (see FIG. 1(b)). The individual halves can be as small as a few dozen of amino acids and, as a result, their production requires a small amount of cellular resources. Split-inteins have been widely studied and characterized due to their extensive usage in various life science disciplines. In fact, they are typically used for fast, reliable and irreversible post-translational modifications. Furthermore, they naturally exist as orthogonal pairs with different kinetic properties, where each pair react among each other without interfering with other pairs. The inventors shall refer to these reactions as “intein-splicing reactions” where molecules containing IntC react with molecules containing IntN to undergo a particular splicing mechanism. Note that split inteins are labeled as active when they are capable of undergoing an intein-splicing reaction; whereas inactive intein complexes denote hetero-dimerized IntC-IntN molecules that are incapable of undergoing any intein-splicing reactions. When two molecules undergo an intein-splicing reaction, the IntN and IntC segments are stoichiometrically inactivated; however, the products of such a reaction may still have other functions such as activating or repressing gene expression.

The basic idea of the inventors' work is to leverage the flexibility offered by split-inteins to design biomolecular controllers capable of achieving Robust Perfect Adaptation (RPA). This flexibility is mainly a consequence of their compatibility with essentially any transcription factor. In fact, the particular structure of the expressed transcription factor involving the choices of the activation domain, dimerization domain, DNA-binding domain and insertion position of the split intein opens the possibilities to a broad spectrum of controllers (see FIG. 1(c)). In fact, this is true not only for transcription factors, but also for other classes of proteins. For instance, it is possible to insert a split-intein between the transmembrane domains of a cytoplasmic domain and a receptor. The resulting intein-splicing reaction would lead to the truncation of the cytoplasmic domain hence preventing signal transduction into the cell.

A first aspect of the invention relates to a method for constant-level expression of an output. The method comprises the steps:
a. providing a cell or a cell-free system, wherein the cell or the cell-free system is capable of expressing a gene encoding the output;
b. inserting an expression system into said cell or cell-free system,
c. exposing said cell or cell-free system to a condition, wherein the expression level of the output is perturbed from a target level to a perturbed level;
d. awaiting an equilibration of the expression level of the output back to the target level.

A second aspect of the invention relates to an expression system for constant-level expression of an output.

A third aspect of the invention relates to a cell comprising the expression system according to the second aspect.

### Terms and Definitions

For purposes of interpreting this specification, the following definitions will apply and whenever appropriate, terms used in the singular will also include the plural and vice versa. In the event that any definition set forth below conflicts with any document incorporated herein by reference, the definition set forth shall control.

The terms “comprising”, “having”, “containing”, and “including”, and other similar forms, and grammatical equivalents thereof, as used herein, are intended to be equivalent in meaning and to be open-ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of embodiments of “consisting essentially of” or “consisting of.”

Where a range of values is provided, it is understood that each intervening value, to the tenth of the unit of the lower limit, unless the context clearly dictates otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

Reference to “about” a value or parameter herein includes (and describes) variations that are directed to that value or parameter per se. For example, description referring to “about X” includes description of “X.”

As used herein, including in the appended claims, the singular forms “a”, “or” and “the” include plural referents unless the context clearly dictates otherwise.

“And/or” where used herein is to be taken as specific recitation of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Unless defined otherwise, all technical and scientific terms used herein have the same meaning as commonly understood by one of ordinary skill in the art (e.g., in cell culture, molecular genetics, nucleic acid chemistry, hybridization techniques and biochemistry, organic synthesis). Standard techniques are used for molecular, genetic, and biochemical methods (see generally, Sambrook et al., Molecular Cloning: A Laboratory Manual, 4th ed. (2012) Cold Spring Harbor Laboratory Press, Cold Spring Harbor, N.Y. and Ausubel et al., Short Protocols in Molecular Biology (2002) 5th Ed, John Wiley & Sons, Inc.) and chemical methods.

Any patent document cited herein shall be deemed incorporated by reference herein in its entirety.

### General Biochemistry: Peptides, Amino Acid Sequences

The term polypeptide in the context of the present specification relates to a molecule consisting of 50 or more amino acids that form a linear chain wherein the amino acids are connected by peptide bonds. The amino acid sequence of a polypeptide may represent the amino acid sequence of a whole (as found physiologically) protein or fragments thereof. The term “polypeptides” and “protein” are used interchangeably herein and include proteins and fragments thereof. Polypeptides are disclosed herein as amino acid residue sequences.

The term peptide in the context of the present specification relates to a molecule consisting of up to 50 amino acids, in particular 8 to 30 amino acids, more particularly 8 to 15 amino acids, that form a linear chain wherein the amino acids are connected by peptide bonds.

Amino acid residue sequences are given from amino to carboxyl terminus. Capital letters for sequence positions refer to L-amino acids in the one-letter code (Stryer, Biochemistry, 3^{rd} ed. p. 21). Lower case letters for amino acid sequence positions refer to the corresponding D- or (2R)-amino acids. Sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

In the context of the present specification, the term dimer refers to a unit consisting of two subunits.

### General Molecular Biology: Nucleic Acid Sequences, Expression

The term gene refers to a polynucleotide containing at least one open reading frame (ORF) that is capable of encoding a particular polypeptide or protein after being transcribed and translated. A polynucleotide sequence can be used to identify larger fragments or full-length coding sequences of the gene with which they are associated. Methods of isolating larger fragment sequences are known to those of skill in the art.

The terms gene expression or expression, or alternatively the term gene product, may refer to either of, or both of, the processes—and products thereof—of generation of nucleic acids (RNA) or the generation of a peptide or polypeptide, also referred to transcription and translation, respectively, or any of the intermediate processes that regulate the processing of genetic information to yield polypeptide products. The term gene expression may also be applied to the transcription and processing of a RNA gene product, for example a regulatory RNA or a structural (e.g. ribosomal) RNA. If an expressed polynucleotide is derived from genomic DNA, expression may include splicing of the mRNA in a eukaryotic cell. Expression may be assayed both on the level of transcription and translation, in other words mRNA and/or protein product.

The term nucleic acid expression vector in the context of the present specification relates to a plasmid, a viral genome or an RNA, which is used to transfect (in case of a plasmid or an RNA) or transduce (in case of a viral genome) a target cell with a certain gene of interest, or -in the case of an RNA construct being transfected- to translate the corresponding protein of interest from a transfected mRNA. The term nucleic acid expression vector in the context of the present specification also relates to a plasmid or an RNA in a cell-free system. For vectors operating on the level of transcription and subsequent translation, the gene of interest is under control of a promoter sequence and the promoter sequence is operational inside the target cell or the cell-free system, thus, the gene of interest is transcribed either constitutively or in response to a stimulus or dependent on the cell's or system's status. In certain embodiments, the viral genome is packaged into a capsid to become a viral vector, which is able to transduce the target cell.

In the context of the present specification, the term expression system refers to a nucleic acid-based encoding of certain genes in a context which allows for expression of the genes. In certain embodiments, each gene of the expression system is preceded (in 5′ to 3′ direction) by a promoter. In certain embodiments, the expression system is composed of one or several expression vectors. In certain embodiments, the expression system is composed of one or several plasmids. In certain embodiments, the expression system is encoded in the genome of the target cell. In certain embodiments, the expression system encoded in a viral particle which then is used to transform the target cell. In certain embodiments, the expression system is composed of DNA. In certain embodiments, the expression system is composed of RNA. In certain embodiments, the expression system additionally comprises enhancer sequences or other regulatory sequences.

In the context of the present specification, the term upregulating the concentration refers to a mechanism or a signal cascade which leads to higher amount of the target polypeptide or peptide (termed “target” hereafter) than the amount which would have been produced without the agent that upregulates the concentration. In certain embodiments, the concentration is upregulated via an increased expression of the target. In certain embodiments, the concentration is upregulated via increased transcription of the target. In certain embodiments, the concentration is upregulated via increased translation of the target. In certain embodiments, the concentration is upregulated via decreased degradation of the target. In certain embodiments, the concentration is upregulated via activation of the target. In certain embodiments, this activation is achieved via a posttranslational modification of the target. In certain embodiments, the concentration is upregulated directly by the agent that upregulates the concentration with the agent being a transcription factor, and the target being under control of that transcription factor. In certain embodiments, the concentration is upregulated via formation of a target protein complex. In certain embodiments, the concentration is upregulated via a combination of the mechanisms described before.

In the context of the present specification, the term downregulating the concentration refers to a mechanism or a signal cascade which leads to lower amount of the target polypeptide or peptide (termed “target” hereafter) than the amount which would have been produced without the agent that downregulates the concentration. In certain embodiments, the concentration is downregulated via a decreased expression of the target. In certain embodiments, the concentration is downregulated via decreased transcription of the target. In certain embodiments, the concentration is downregulated via decreased translation of the target. In certain embodiments, the concentration is downregulated via increased degradation of the target. In certain embodiments, the concentration is downregulated via inactivation of the target. In certain embodiments, this inactivation is achieved via a posttranslational modification of the target. In certain embodiments, the concentration is downregulated directly by the agent that downregulates the concentration with the agent being a transcription repressor, and the target being under control of that transcription repressor. In certain embodiments, the concentration is downregulated via sequestration of a target protein complex. In certain embodiments, the concentration is downregulated via a combination of the mechanisms described before.

In the context of the present specification, the terms upregulating or downregulating the concentration do not necessarily mean that all mentioned agents only have the respective effect. It rather means that the summed effect of all mentioned agents under steady-state conditions have the respective effect.

As an example, if the second and the optional first splice-product downregulate the output, it could be that an intermediate splice-product has an upregulating effect on the output. It could also be that e.g. the first splice-product has an upregulating effect, but the second splice-product has an even stronger downregulating effect. It could also be that a single species, as e.g. the first splice-product, has a mixed effect on the concentration of the output, and partially upregulates and partially downregulates the concentration of the output. Yet, in this example, the summed effect of the first and the second splice-product under steady-state conditions is an upregulation of the output.

In the context of the present specification, the term steady-state condition refers to the maintenance of constant internal concentrations of molecules and ions. A continuous flux of mass and energy results in the constant synthesis and breakdown of molecules via chemical reactions of biochemical pathways. In the present context, steady-state refers to a situation in which the output expression level is not disturbed via an external stimulus, or in which the disturbance has already been re-equilibrated.

In the context of the present specification, the term effector refers to a peptide or polypeptide which (optionally in combination with other effectors) has an impact or influence on the concentration of a target protein, i.e. the output. In certain embodiments, each effector (which is present) is composed of or comprises one or several protein domain(s). In certain particular embodiments, each effector (which is present) comprises one or several transcription factor domain(s). Before the trans-splicing reaction of the N-intein and the C-intein occurs, the first and the second effectors are in close proximity, and the third and the fourth effectors are in close proximity. After the trans-splicing reaction of the N-intein and the C-intein has occurred, the first and the fourth effectors are in close proximity, and the second and the third effectors are in close proximity. Depending on their proximity, the different domains interact in a different way. In certain embodiments, several trans-splicing reactions occur via several N-inteins and/or several C-inteins, but the same rules as for single inteins apply.

In the context of the present specification, the term intein refers to a split-intein. Split inteins are split into two fragments called N-intein (I_{N}) and C-intein (I_{C}). Once translated, the intein fragments bind to each other allowing them to assemble intermolecularly into a fully functional protein splicing unit, which catalyzes protein splicing in trans. For further details see Sarmiento and Camarero (Curr Protein Pept Sci. 2019; 20(5): 408-424.).

In the context of the present specification, the term trans-splicing refers to excision of the N-intein and the C-intein from their polypeptide chain, and rejoining of the (poly)peptide N-terminal of the N-intein with the (poly)peptide C-terminal of the C-intein. The N-intein forms a non-covalent protein complex with the C-intein when brought in close proximity. This protein complex performs the trans-splicing reaction.

In the context of the present specification, the term rejoining refers to the formation of a covalent peptide bond. An N-terminal amino acid of one protein is linked to a C-terminal amino acid of the other protein via an amide bond between the protein backbones of these proteins.

In the context of the present specification, the term perturbation refers to a deviation of a concentration level from the target level to a perturbed level. In certain embodiments, an external stimulus leads to the perturbation of the expression level.

In the context of the present specification, the term cell refers to a prokaryotic cell or a eukaryotic cell.

In the context of the present specification, the term cell-free system refers to an in vitro system derived from a prokaryotic cell or a eukaryotic cell, wherein the system contains the native transcription, translation, and metabolic machineries required to achieve gene expression.

In the context of the present specification, the term loop region refers to a longer, extended or irregular loop inside a protein structure without fixed internal hydrogen bonding.

The term proportionally regulating in the context of the present specification relates to a regulation, wherein an increase of the amount of the regulator leads to an increase of the amount of the regulated species, wherein the magnitude of the increasing amounts is proportional. This means: the higher the increase of the regulator, the higher is the increase of the regulated species.

The term anti-proportionally regulating in the context of the present specification relates to a regulation, wherein an increase of the amount of the regulator leads to a decrease of the amount of the regulated species, wherein the magnitude of the increasing amount is anti-proportional to the decreasing amount. This means: the higher the increase of the regulator, the stronger is the decrease of the regulated species.

In the context of the present invention, the second splice-product and the optional first splice-product have a weaker, neutral, or opposite effect on the concentration of the output in comparison to the effect of the controller. This means that if
a. the controller upregulates the concentration of the output:
   The second splice-product and the optional first splice-product in sum do not upregulate the concentration of the output to the same extent as the controller does. Thus, the second splice-product and the optional first splice-product in sum
      i. weakly upregulate the concentration of the output (significantly weaker than the controller upregulates the output); or
      ii. have no (a neutral) effect on the concentration of the output; or
      iii. downregulate the concentration of the output.
b. the controller downregulates the concentration of the output:
   The second splice-product and the optional first splice-product in sum do not downregulate the concentration of the output to the same extent as the controller does. Thus, the second splice-product and the optional first splice-product in sum
      i. weakly downregulate the concentration of the output (significantly weaker than the controller downregulates the output); or
      ii. have no (a neutral) effect on the concentration of the output; or
      iii. upregulate the concentration of the output.

This feature of the second splice-product and the optional first splice-product is important in achieving perfect-robust-adaption. The idea is that the controller has a certain effect on the concentration of the output, and that this effect is lowered or even reversed when the controller reacts with the anti-controller to form the second splice-product and the optional first splice-product.

The step of awaiting an equilibration of the expression level of the output back to the target level is to be understood as follows: The proposed intein-based controller circuits realize what is in control theory referred to as “Integral Feedback Control”—one of the most predominant control strategies in industry. This type of feedback is more stringent than simple negative feedback where the controller's task is to only oppose the variation of the output of interest to be controlled. In contrast, the integral feedback controller, realized with split inteins, determines the error or, more specifically, the deviation of the output from the desired setpoint and feeds back the mathematical integral as the control action that steers the output to the desired level. It is precisely this mathematical determination of the integral of the error that endows the system with the robust perfect adaptation property that guarantees the equilibration of the output concentration level back to its pre-perturbed state. These mathematical determinations are encoded in the various reactions occurring between the controller and anti-controller species. In certain embodiments, the controller is constitutively expressed to encode for the desired setpoint level. In certain embodiments, the expression of the anti-controller is induced by the output to “sense” its abundance. In certain embodiments, the intein-splicing reaction between the controller and anti-controller encodes for the comparison between setpoint and output concentration to compute the error and its mathematical integral. In certain embodiments, the controller molecules that are not sequestered by the anti-controller induces the expression of the output, and thus effectively closes the loop by feeding back the mathematical integral. In certain embodiments, the spliced products aid in enhancing the transient dynamic performance (e.g. speeding up the response, damping oscillations, reducing overshoots, etc.).

### DETAILED DESCRIPTION OF THE INVENTION

Inteins were first discovered in 1990, while split inteins have been known since 1998. However, some split inteins have specific requirements that make them challenging to use in synthetic biology. In 2009, the GP41-1 split intein was discovered and characterized as one of the best split inteins to date. This particular split intein pair was utilized in our invention.

Prior to our application for the present invention, there was a six-year overlap between the discovery of inteins and the idea of the antithetic integral feedback. Despite this overlap, neither of the successful applications during that period utilized inteins. The parts chosen in the previous implementations were limited to their respective host organisms and were not easily transferable to bacteria or eukaryotes. However, the intein-based approach presented here seems to outperform both designs in their respective organisms.

Feedback is an important concept in biology and is widely utilized. Integral feedback is particularly powerful due to its potential for achieving robust perfect adaptation (RPA). The antithetic integral feedback motif is the simplest motif that achieves RPA under stochastic conditions, which are omnipresent in cells. Achieving RPA in cells is a highly sought-after goal.

There are several reasons why neither control theorists nor synthetic biologists have utilized intein-based solutions for the antithetic integral feedback motif. The first introduction of the antithetic integral feedback motif to the scientific community emphasized the need for the annihilation of the controller molecules (Briat et al., 2016. Cell Systems 2 (1): 15-26.). While the work was theoretical, the authors acknowledged that physical annihilation, similar to matter and antimatter, was not feasible. They proposed a more realistic irreversible binding of Z1 and Z2, which mutually inhibited their function for biological implementations. Since then, the need for sequestration, annihilation, and mutual inactivation has been repeatedly emphasized in publications.

Utilizing inteins instead for the antithetic motif is not an obvious choice. Previous applications focused on finding molecules that bind as strongly as possible. Split inteins on the other hand perform protein splicing, generating three proteins, with two of them remaining as a heterodimer. Additionally, the sequestering molecules used in previous applications (mRNA for translation and sigma factors for transcription) directly participated in the network's actuation. Split inteins, on the other hand, cannot inherently participate in such catalytic reactions, leading to a fundamental separation between the actuating and sequestering domains. Furthermore, it was previously believed that inactivation had to be absolute to avoid interference with the performance. However, we have demonstrated that the complex does not need to interfere with robust perfect adaptation properties. In fact, by inverting the function, it can be used to improve these properties.

Nevertheless, it is possible to implement the traditional antithetic integral feedback motif using inteins. By carefully selecting the actuating protein, choosing a suitable insertion site for the intein, and combining it with the protein bond-breaking properties of inteins, it is possible to generate three products, none of which retain any function, as demonstrated in our examples. However, moving from strong binding partners, which act simultaneously as actuators to separate functional domains with irreversible protein cleavage through protein splicing represents a non-trivial and fundamentally new approach. These advancements required creative and innovative thinking combined with the development of extensive theory culminating in the generation of a mathematical theorem.

### Advantages of Using Split-Inteins

The stoichiometric “sequestration” reaction—functioning as the exclusive (or practically predominant) degradation pathway for the involved molecular pair—forms the core of biomolecular integral controllers that ensure Robust Perfect Adaptation (RPA). Split inteins are excellent candidates for implementing this reaction efficiently due to several direct and indirect reasons.

### Direct Reasons:

1. Split inteins exist in pairs, making them well-suited for this task.
2. They implement an irreversible “sequestration” reaction, a necessity in practical circumstances (where biomolecular dilution is non-zero).
3. The splicing of inteins is intrinsically stoichiometric.
4. They are available in a plethora of orthogonal pairs, enabling their deployment in multiple independent integral controllers.
5. Inteins offer high flexibility in implementing “sequestration” reactions to control diverse genetic systems.

### Indirect Reasons:

1. Owing to their small size, inteins impose a minimal burden and do not significantly impair proteins when synthetically inserted.
2. They are genetically encodable proteins, offering convenient incorporation into biological systems.
3. The flexibility of inteins offer diverse designs that leverage the products of the splicing reaction in enhancing dynamic performance while preserving RPA.

### Downsides of TF:

Natural transcription factors/inhibitor pairs are rare
They have reversible binding
Exogenous sigma factors have a negative impact on the cellular fitness
   Downsides of mRNA:
mRNA is in general less stable than proteins
double stranded mRNA can trigger cellular defence mechanisms as they are often associated with viral infections

A first aspect of the invention relates to a method for constant-level expression of an output. The method comprises the steps:
a. providing a cell or a cell-free system, wherein the cell or the cell-free system is capable of expressing a gene encoding the output under control of a first promoter;
b. inserting an expression system into said cell or cell-free system,
c. exposing said cell or cell-free system to a condition, wherein the expression level of the output is perturbed from a target level to a perturbed level;
d. awaiting an equilibration of the expression level of the output back to the target level.

The first, second, third, and fourth effector are peptides or polypeptides, and the first and second intein, the controller, and the anti-controller are polypeptides. The output is a peptide, a polypeptide, or an mRNA.

A second aspect of the invention relates to an expression system for constant-level expression of an output which is under control of a first promoter.

The expression system of the first and the second aspect comprises:
i. a gene encoding a controller under control of a second promoter, wherein the controller comprises from N to C-terminus:
   I. an optional first effector;
   II. a first split-intein; and
   III. an optional second effector;
ii. a gene encoding an anti-controller under control of a third promoter, wherein the anti-controller comprises from N to C-terminus:
   I. an optional third effector;
   II. a second split-intein; and
   III. an optional fourth effector.

Thus, the controller comprises
a) a first effector, a first split-intein, and a second effector; or
b) a first effector, and a first split-intein; or
c) a first split-intein, and a second effector; or
d) only a first split-intein, and, the anti-controller comprises
i.) a third effector, a second split-intein, and a fourth effector; or
ii.) a third effector, and a second split-intein; or
iii.) a second split-intein, and a fourth effector; or
iv.) only a second split-intein;
   with the proviso that at least one of the optional effectors is present (the option of d) the controller comprises only a first split-intein cannot be combined with the option of iv.) the anti-controller comprises only a second split-intein).

One of the first split-intein and the second split-intein is an N-intein and the other one is a C-intein. Thus, either the first split-intein is an N-intein, and the second split-intein is a C-intein, or the first split-intein is a C-intein, and the second split-intein is an N-intein.

The controller and the anti-controller when being in close proximity are capable of undergoing a trans-splicing reaction. The trans-splicing reaction depending on whether the first split-intein (alternative a) or the second split-intein (alternative b) is the N-intein comprises the following steps:
Alternative a): the first split-intein is an N-intein, and the second split-intein is a C-intein: The first split-intein is excised from the controller, and the second split-intein is excised from the anti-controller. The first effector if present is rejoined with the fourth effector if present yielding a first splice-product. Thus, the first splice-product consists of
   the first effector, and the fourth effector, or
   only of the first effector, or
   only of the fourth effector, or
   is not present at all.
A non-covalently associated protein complex is assembled consisting of the N-intein covalently bound to the second effector if present with the C-intein covalently bound to the third effector if present yielding a second splice-product. Thus, the second splice-product is a non-covalently associated protein complex which consists of
   the N-intein covalently bound to the second effector, and the C-intein covalently bound to the third effector, or
   the N-intein covalently bound to the second effector, and the C-intein,
   the N-intein, and the C-intein covalently bound to the third effector,
   the N-intein, and the C-intein.
The only proviso is that one of the optional effectors is present, thus if the first splice-product is not present at all, the second splice-product cannot consist only of the N-intein and the C-intein.
Alternative b): the first split-intein is a C-intein, and the second split-intein is an N-intein: The first split-intein is excised from the controller, and the second split-intein is excised from the anti-controller. The second effector if present is rejoined with the third effector if present yielding a first splice-product. Thus, the first splice-product consists of
   the second effector, and the third effector, or
   only of the second effector, or
   only of the third effector, or
   is not present at all.
A non-covalently associated protein complex is assembled consisting of the N-intein covalently bound to the fourth effector if present with the C-intein covalently bound to the first effector if present yielding a second splice-product. Thus, the second splice-product is a non-covalently associated protein complex which consists of
   the N-intein covalently bound to the fourth effector, and the C-intein covalently bound to the first effector, or
   the N-intein covalently bound to the fourth effector, and the C-intein,
   the N-intein, and the C-intein covalently bound to the first effector,
   the N-intein, and the C-intein.
The only proviso is that one of the optional effectors is present, thus if the first splice-product is not present at all, the second splice-product cannot consist only of the N-intein and the C-intein.

The anti-controller is capable of downregulating the controller stoichiometrically via undergoing the trans-splicing reaction. This is because the controller and the anti-controller react in the trans-splicing reaction yielding the second splice-product and optionally the first splice-product.

The second splice-product and the optional first splice-product have a weaker, neutral, or opposite effect on the concentration of the output in comparison to the effect of the controller. Thus, the effect of the controller on the output is decreased or even reversed when the controller reacts with the anti-controller.

Constant-level expression of the output is achieved via one of the following four alternatives of a steady-state route (also called architecture or topology).

### Alternative (1) of a Steady-State Route:

The controller upregulates or is capable of upregulating the concentration of the output. The output upregulates or is capable of upregulating the concentration of the anti-controller. The second splice-product and the optional first splice-product are not capable of upregulating the concentration of the output to the extent as the controller (weaker upregulation, neutral effect, or downregulation). In certain embodiments, the anti-controller additionally downregulates or is capable of downregulating the concentration of the output.
   Alternative (2) of a steady-state route:
The controller downregulates or is capable of downregulating the concentration of the output.

The output downregulates or is capable of downregulating the concentration of the anti-controller. The second splice-product and the optional first splice-product are not capable of downregulating the concentration of the output to the extent as the controller (weaker downregulation, neutral effect, or upregulation). In certain embodiments, the anti-controller additionally upregulates or is capable of upregulating the concentration of the output.

### Alternative (3) of a Steady-State Route:

The controller upregulates or is capable of upregulating the concentration of the output. The output downregulates or is capable of downregulating the concentration of the controller. The second splice-product and the optional first splice-product are not capable of upregulating the concentration of the output to the extent as the controller (weaker upregulation, neutral effect, or downregulation). In certain embodiments, the anti-controller additionally downregulates or is capable of downregulating the concentration of the output.

### Alternative (4) of a Steady-State Route:

The controller downregulates or is capable of downregulating the concentration of the output. The output upregulates or is capable of upregulating the concentration of the controller. The second splice-product and the optional first splice-product are not capable of downregulating the concentration of the output to the extent as the controller (weaker downregulation, neutral effect, or upregulation). In certain embodiments, the anti-controller additionally upregulates or is capable of upregulating the concentration of the output.

In certain embodiments, the controller comprises a further first intein and/or the anti-controller comprises a further second intein and the trans-splicing reaction is executed (or is executable) multiple times finally yielding the second splice-product and optionally the first splice-product. For an example, see FIGS. 10 and 11.

In certain embodiments, the first intein is inserted into the controller in a loop region of the first and second effector. In certain embodiments, the second intein is inserted into the anti-controller in a loop region of the third and fourth effector.

In certain embodiments,
the first effector, and/or
the second effector, and/or
the third effector, and/or
the fourth effector
   comprise a (one or several) domain(s) of a transcription factor. In certain embodiments, the first effector comprises a (one or several) domain(s) of a transcription factor. In certain embodiments, the second effector comprises a (one or several) domain(s) of a transcription factor. In certain embodiments, the third effector comprises a (one or several) domain(s) of a transcription factor. In certain embodiments, the fourth effector comprises a (one or several) domain(s) of a transcription factor.

In certain embodiments,
the controller is a non-dimerizing transcription factor (TF) (particularly a zinc-finger-based transcription factor) modified by insertion of a C-intein;
the first effector is a DNA-binding domain of the TF;
the second effector is an activation domain of the TF;
the anti-controller is an N-intein;
the third and the fourth effectors are not present.

In certain embodiments,
the controller is a dimerizing transcription factor (TF) (particularly a tetR-based transcription factor) modified by insertion of a C-intein;
the first effector consists of
   a DNA-binding domain of the TF;
   an N-terminal part of a dimerization domain of the TF;
the second effector consists of
   a C-terminal part of a dimerization domain of the TF;
   an activation domain of the TF;
the anti-controller is an N-intein;
the third and the fourth effectors are not present.

In certain embodiments,
the controller is a dimerizing transcription factor (TF) (particularly a tTA-based transcription factor) modified by insertion of a C-intein;
the first effector consists of
   a DNA-binding domain of the TF;
   a dimerization domain of the TF;
the second effector is an activation domain of the TF;
the anti-controller is an N-intein;
the third and the fourth effectors are not present.

In certain embodiments,
the controller is a dimerizing transcription factor (TF) (particularly a GAL4-based transcription factor) modified by insertion of a C-intein;
the first effector is a DNA-binding domain of the TF;
the second effector consists of
   a dimerization domain of the TF;
   an activation domain of the TF;
the anti-controller is an N-intein;
the third and the fourth effectors are not present.

In certain embodiments,
the controller is an activation domain modified by fusion of a C-intein;
the second effector is an activation domain;
the first effector is not present;
the anti-controller is an N-intein;
the third and the fourth effectors are not present;
the expression system additionally comprises a co-controller, wherein the co-controller comprises from N-terminus to C-terminus
   a DNA-binding domain of the TF;
   a dimerization domain of the TF;
   an inactive N-intein.

In certain embodiments, the output is a cytokine.

In certain embodiments,
the first effector, and/or
the second effector, and/or
the third effector, and/or
the fourth effector
   comprise a domain of an inhibitor of said cytokine. In certain embodiments, the inhibitor is an antibody. In certain embodiments,
for case (1): the output is capable of regulating the concentration of the anti-controller proportionally; or
for case (2): the output is capable of regulating the concentration of the anti-controller anti-proportionally; or
for case (3): the output is capable of regulating the concentration of the controller anti-proportionally; or
for case (4): the output is capable of regulating the concentration of the controller proportionally.

In certain embodiments,
for case (1) or (2): the target expression level of the output is tunable via adjusting a production rate of the controller and/or via adjusting a ratio of a production rate of the anti-controller to an output expression level; or
for case (3) or (4): the target expression level of the output is tunable via adjusting a production rate of the anti-controller and/or via adjusting a ratio of a production rate of the controller to an output expression level.

In certain embodiments,
for case (1) or (2): the target expression level of the output is tunable via adjusting a production rate of the controller and/or via adjusting a production rate of the anti-controller, wherein the production rate of the anti-controller is a function of the output expression level; or
for case (3) or (4): the target expression level of the output is tunable via adjusting a production rate of the anti-controller and/or via adjusting a production rate of the controller, wherein the production rate of the controller is a function of the output expression level.

The function of the production rate of the (anti-)controller to the output may be non-linear, as shown in FIG. 20.

In certain embodiments, for cases (1) and (2): the target expression level of the output is tunable. The term tunable means that the target expression level can be set to a defined level. This level is determined by both the production rate of the controller (set to a constant value, e.g. by constitutive expression) and by the production rate of the anti-controller (made to be dependent on the expression level of the output via a mechanism designed for that purpose). In practice, this dependence is achieved (for example) by driving the expression of the anti-controller with a transcription factor (activator or repressor) that is either fused to the output or is a proxy for it. In this way, the production rate of the anti-controller functions as a sensor of the output expression level. The defined expression level of the output will be that which results in equal production rates of the anti-controller and the controller. For example, if the production rate of the anti-controller is linearly proportional to the expression level of the output, then the defined expression level of the output will be the ratio of the controller production rate and the proportionality constant of the anti-controller production rate. Thus, the target expression level of the output is tunable by adjusting either the production rate of the controller and/or via adjusting the production rate of the anti-controller. In certain embodiments, for cases (3) and (4): the target expression level of the output is tunable. The term tunable means that the target expression level can be set to a defined level. This level is determined by both the production rate of the anti-controller (set to a constant value, e.g. by constitutive expression) and by the production rate of the controller (made to be dependent on the expression level of the output via a mechanism designed for that purpose). In practice, this dependence is achieved (for example) by driving the expression of the controller with a transcription factor (activator or repressor) that is either fused to the output or is a proxy for it. In this way, the production rate of the controller functions as a sensor of the output expression level. The defined expression level of the output will be that which results in equal production rates of the controller and the anti-controller. For example, if the production rate of the controller is linearly proportional to the expression level of the output, then the defined expression level of the output will be the ratio of the anti-controller production rate and the proportionality constant of the controller production rate. Thus, the target expression level of the output is tunable by adjusting either the production rate of the controller and/or via adjusting the production rate of the anti-controller.

A third aspect of the invention relates to a cell comprising the expression system according to the second aspect.

Wherever alternatives for single separable features such as, for example, an isotype protein or ligand type or network topology are laid out herein as “embodiments”, it is to be understood that such alternatives may be combined freely to form discrete embodiments of the invention disclosed herein.

Thus, any of the alternative embodiments for an isotype protein may be combined with any of the alternative embodiments of ligand and these combinations may be combined with any network topology mentioned herein.

The invention further encompasses the following items: Items
0. A method for constant-level expression of an output, said method comprising the steps:
   a. providing a cell and/or a cell-free system, wherein the system is capable of acting positively or negatively on the levels of a specific output of interest, which can be part of a larger circuit (regulated network) inside and/or outside of said cell or cell-free system.
   b. inserting an expression controller system into said cell or cell-free system, providing closed-loop stability, wherein the expression controller system is composed of three subnetworks: N-class, C-class, and S-class.
      i. N-class.
         I. Every protein and protein complex in this subnetwork contains at least one functional IntN of a split-intein which can pair with a functional IntC from the C-class to undergo an intein-splicing reaction. The said IntNs can be attached to or embedded inside and/or between one or multiple effector molecules.
         II. At least one protein within this class is produced either constitutively or inducibly by the controlled output species. Their expression rates can be positively or negatively influenced by the controlled output species.
         III. Proteins and protein complexes within this class are allowed to be converted to one another while conserving the number of active IntN segments.
         IV. Proteins and protein complexes within this class are allowed to bind with other controller species while conserving the number of active IntN segments.
         V. At least one protein belongs to this class.
      ii. C-class
         I. Every protein and protein complex in this subclass contains at least one functional IntC of a split-intein which can pair with the functional IntN from the N class to undergo an intein-splicing reaction. The said IntCs can be attached to or embedded inside and/or between one or multiple effector molecules.
         II. At least one protein within this class is produced either constitutively or inducibly by the controlled output species. Their expression rates can be positively or negatively influenced by the controlled output species.
         III. Proteins and protein complexes within this class are allowed to be converted to one another while conserving the number of active IntC segments.
         IV. Proteins and protein complexes within this class are allowed to bind with other controller species while conserving the number of active IntN segments.
         V. At least one protein belongs to this class.
      iii. S-class
         I. All protein and protein complexes, which do not belong to the C- and N-classes, belong to the S-class.
1. A method for constant-level expression of an output, said method comprising the steps:
   a. providing a cell or a cell-free system, wherein the cell or the cell-free system is capable of expressing a gene encoding the output;
   b. inserting an expression system into said cell or cell-free system, wherein the expression system comprises:
      i. a gene encoding a controller, wherein the controller comprises:
         I. an optional first effector;
         II. a first split-intein; and
         III. an optional second effector;
      ii. a gene encoding an anti-controller, wherein the anti-controller comprises:
         I. an optional third effector;
         II. a second split-intein; and
         III. an optional fourth effector;
      wherein
         at least one of the optional effectors is present;
         one of the first split-intein and the second split-intein is an N-intein and the other one is a C-intein;
         the controller and the anti-controller are capable of undergoing a trans-splicing reaction via the steps:
         excising the first split-intein from the controller, and excising the second split-intein from the anti-controller, and
         a) if the first split-intein is an N-intein, and the second split-intein is a C-intein:
         rejoining
         the first effector if present with
         the fourth effector if present
         yielding a first splice-product;
         assembling a protein complex of
         the N-intein
         bound to the second effector if present
         with
         the C-intein
         bound to the third effector if present
         yielding a second splice-product;
         b) if the first split-intein is a C-intein, and the second split-intein is an N-intein:
         rejoining
         the second effector if present with
         the third effector if present
         yielding a first splice-product;
         assembling a protein complex of
         the N-intein
         bound to the fourth effector if present
         with
         the C-intein
         bound to the first effector if present
         yielding a second splice-product;
         wherein at least the second splice-product is yielded;
         the anti-controller is capable of downregulating the controller stoichiometrically via undergoing the trans-splicing reaction;
         the second splice-product and the optional first splice-product have a weaker, neutral, or opposite effect on the concentration of the output in comparison to the effect of the controller;
         constant-level expression of the output is achieved via a route selected from the group consisting of:
      (1)
         the controller upregulates the concentration of the output;
         the output upregulates the concentration of the anti-controller;
      (2)
         the controller downregulates the concentration of the output;
         the output downregulates the concentration of the anti-controller;
      (3)
         the controller upregulates the concentration of the output;
         the output downregulates the concentration of the controller;
      (4)
         the controller downregulates the concentration of the output;
         the output upregulates the concentration of the controller;
   c. exposing said cell or cell-free system to a condition, wherein the expression level of the output is perturbed from a target level to a perturbed level;
   d. equilibration of the expression level of the output back to the target level;
wherein the first, second, third, and fourth effector are peptides or polypeptides, and the first and second intein, the controller, and the anti-controller are polypeptides, and the output is a peptide, a polypeptide, or an mRNA.
2. An expression system for constant-level expression of an output, said system comprising:
   a. a gene encoding a controller, wherein the controller comprises:
      i.) an optional first effector;
      ii.) a first split-intein; and
      iii.) an optional second effector;
   b. a gene encoding an anti-controller, wherein the anti-controller comprises:
      i.) an optional third effector;
      ii.) a second split-intein; and
      iii.) an optional fourth effector;
   wherein
      at least one of the optional effectors is present;
      one of the first split-intein and the second split-intein is an N-intein and the other one is a C-intein;
      the controller and the anti-controller are capable of undergoing a trans-splicing reaction via the steps:
      excising the first split-intein from the controller, and excising the second split-intein from the anti-controller, and
         a) if the first split-intein is an N-intein, and the second split-intein is a C-intein:
         rejoining
         the first effector if present with
         the fourth effector if present
         yielding a first splice-product;
         assembling a protein complex of
         the N-intein
         bound to the second effector if present
         with
         the C-intein
         bound to the third effector if present
         yielding a second splice-product;
         b) if the first split-intein is a C-intein, and the second split-intein is an N-intein:
         rejoining
         the second effector if present with
         the third effector if present
         yielding a first splice-product;
         assembling a protein complex of
         the N-intein
         bound to the fourth effector if present
         with
         the C-intein
         bound to the first effector if present
         yielding a second splice-product;
         wherein at least the second splice-product is yielded;
      the anti-controller is capable of downregulating the controller stoichiometrically via undergoing the trans-splicing reaction;
      the second splice-product and the optional first splice-product have a weaker, neutral, or opposite effect on the concentration of the output in comparison to the effect of the controller;
      a route via which constant-level expression of the output is achieved is selected from the group consisting of:
      (1)
         the controller is capable of upregulating the concentration of the output;
         the output is capable of upregulating the concentration of the anti-controller;
      (2)
         the controller is capable of downregulating the concentration of the output;
         the output is capable of downregulating the concentration of the anti-controller;
      (3)
         the controller is capable of upregulating the concentration of the output;
         the output is capable of downregulating the concentration of the controller;
      (4)
         the controller is capable of downregulating the concentration of the output;
         the output is capable of upregulating the concentration of the controller,
   wherein the first, second, third, and fourth effector are peptides or polypeptides, and the first and second intein, the controller, and the anti-controller are polypeptides, and the output is a peptide, a polypeptide, or an mRNA.
3. The method according to item 1 or the expression system according to item 2, wherein the controller comprises a further first intein and/or the anti-controller comprises a further second intein and the trans-splicing reaction is executed multiple times finally yielding the second splice-product and optionally the first splice-product.
4. The method according to item 1 or 3 or the expression system according to item 2 or 3, wherein
   the controller is capable of upregulating the concentration of the output;
   the second splice-product and the optional first splice-product are not capable of upregulating the concentration of the output to the extent as the controller;
   the output is capable of upregulating the concentration of the anti-controller.
5. The method according to any one of the preceding items 1 or 3 to 4 or the expression system according to any one of the preceding items 2 to 4, wherein for case (1) that
   the controller is capable of upregulating the concentration of the output;
   the output is capable of upregulating the concentration of the anti-controller; additionally, the anti-controller is capable of downregulating the concentration of the output.
6. The method according to item 1 or 3 or the expression system according to item 2 or 3, wherein
   the controller is capable of downregulating the concentration of the output;
   the second splice-product and the optional first splice-product are not capable of downregulating the concentration of the output to the extent as the controller;
   the output is capable of downregulating the concentration of the anti-controller.
7. The method according to any one of the preceding items 1 or 3 or 6 or the expression system according to any one of the preceding items 2 to 3 or 6, wherein for case (2) that
   the controller is capable of downregulating the concentration of the output;
   the output is capable of downregulating the concentration of the anti-controller; additionally, the anti-controller is capable of upregulating the concentration of the output.
8. The method according to item 1 or 3 or the expression system according to item 2 or 3, wherein
   the controller is capable of upregulating the concentration of the output;
   the second splice-product and the optional first splice-product are not capable of upregulating the concentration of the output to the extent as the controller;
   the output is capable of downregulating the concentration of the controller.
9. The method according to any one of the preceding items 1 or 3 or 8 or the expression system according to any one of the preceding items 2 or 3 or 8, wherein for case (3) that
   the controller is capable of upregulating the concentration of the output;
   the output is capable of downregulating the concentration of the controller; additionally, the anti-controller is capable of downregulating the concentration of the output.
10. The method according to item 1 or 3 or the expression system according to item 2 or 3, wherein
   the controller is capable of downregulating the concentration of the output;
   the second splice-product and the optional first splice-product are not capable of downregulating the concentration of the output to the extent as the controller;
   the output is capable of upregulating the concentration of the controller.
11. The method according to any one of the preceding items 1 or 3 or 10 or the expression system according to any one of the preceding items 2 or 3 or 10, wherein for case (4) that
   the controller is capable of downregulating the concentration of the output;
   the output is capable of upregulating the concentration of the controller;
   additionally, the anti-controller is capable of upregulating the concentration of the output.
12. The method according to any one of the preceding items 1 or 3 to 11 or the expression system according to any one of the preceding items 2 to 11, wherein the first intein is inserted into the controller in a loop region of the first and second effector and/or the second intein is inserted into the anti-controller in a loop region of the third and fourth effector.
13. The method according to any one of the preceding items 1 or 3 to 12 or the expression system according to any one of the preceding items 2 to 12, wherein
   the first effector, and/or
   the second effector, and/or
   the third effector, and/or
   the fourth effector
   comprise a domain of a transcription factor.
14. The method according to item 13 or the expression system according to item 13, wherein
the controller is a non-dimerizing transcription factor (TF) modified by insertion of a C-intein;
the first effector is a DNA-binding domain of the TF;
the second effector is an activation domain of the TF;
the anti-controller is an N-intein;
the third and the fourth effectors are not present.
15. The method according to item 13 or the expression system according to item 13, wherein
   the controller is a dimerizing transcription factor (TF) modified by insertion of a C-intein;
   the first effector consists of
      a DNA-binding domain of the TF;
      an N-terminal part of a dimerization domain of the TF;
   the second effector consists of
      a C-terminal part of a dimerization domain of the TF;
      an activation domain of the TF;
   the anti-controller is an N-intein;
   the third and the fourth effectors are not present.
16. The method according to item 13 or the expression system according to item 13, wherein
   the controller is a dimerizing transcription factor (TF) modified by insertion of a C-intein;
   the first effector consists of
      a DNA-binding domain of the TF;
      a dimerization domain of the TF;
   the second effector is an activation domain of the TF;
   the anti-controller is an N-intein;
   the third and the fourth effectors are not present.
17. The method according to item 13 or the expression system according to item 13, wherein
   the controller is a dimerizing transcription factor (TF) modified by insertion of a C-intein;
   the first effector is a DNA-binding domain of the TF;
   the second effector consists of
      a dimerization domain of the TF;
      an activation domain of the TF;
   the anti-controller is an N-intein;
   the third and the fourth effectors are not present.
18. The method according to item 13 or the expression system according to item 13, wherein
   the controller is an activation domain modified by fusion of a C-intein;
   the second effector is an activation domain;
   the first effector is not present;
   the anti-controller is an N-intein;
   the third and the fourth effectors are not present;
   the expression system additionally comprises a co-controller, wherein the co-controller comprises
      a DNA-binding domain of the TF;
      a dimerization domain of the TF;
      an inactive N-intein.
19. A cell comprising the expression system according to any one of the preceding items 2 to 18. The invention is further illustrated by the following examples and figures, from which further embodiments and advantages can be drawn. These examples are meant to illustrate the invention but not to limit its scope.

### DESCRIPTION OF THE FIGURES

FIG. 1 shows exploiting Intein splicing for building genetic controllers. (a) The basic antithetic integral feedback motif. The arbitrary network to the left represents the regulated network. The input and output molecules of this network are denoted by X₁ and X_{L}, respectively. The feedback controller senses the output X_{L} and actuates the input X₁ accordingly. The network in the middle represents the basic antithetic integral feedback motif. The controller molecule Z₁ is constitutively produced, while Z₂ is catalytically produced by the output X_{L}. Furthermore, Z₁ and Z₂ sequester each other to produce an inert complex Ø that has no effect on the closed-loop dynamics. The plot to the right demonstrates that integral controllers achieve Robust Perfect Adaptation (RPA): when a disturbance is injected in the presence of the integral controller, the output concentration level transiently deviates from the desired setpoint but then restores back to it. (b) Possible split-intein reactions. The protein trans-splicing of split inteins is achieved through the specific binding of both intein subunits, which leads to conformational change and chemical bonds shifting on the junction site. The splicing process can be summarized in several essential steps. (i) The precursor 1 containing Intein-N and precursor 2 containing Intein-C are individual proteins. (ii) Intein-N and Intien-C dimerize and reconstitute the active intein domain, which undergoes N—S acyl shift and trans-thioesterification reaction to form a branched intermediate. (iii) The last residue of intein-C forms a succinimide ring, effectively cleaving intein-C from the branched intermediate. (iv) Finally, the end products of protein trans-splicing contain a single-chain protein (comprising the N-terminal of precursor 1 and the C-terminal of precusor2) and a heterodimer (comprising inactive Intein N and Intein-C). Depending on the orientation of the educts (Precursor 1 and Precursor 2), there are several usages of intein-splicing, including sequence ligation, deletion, exchange, or intein cleavage. (c) Examples of realizations of the sequestration reaction via intein splicing. The sequestration reaction lies at the heart of the integral controller shown in panel (a). Based on the capability of split inteins to irreversibly modify the protein sequence or structure, there are several potential implementations to realize the “sequestration reaction” of the antithetic integral controller shown in panel(a). (i) Irreversible Competitive Binding: The heterodimer comprised of the DNA-binding domain and activation domain is irreversibly separated by an intein-mediated removal of intein-N from the DNA-binding domain (ii) Conversion: the target transcription activator is converted to a repressor by an intein-mediated exchange of the activation domain with a repression domain. (iii) Inactivation: the target transcription factor is inactivated by an intein-mediated deletion of its partial sequence. (iv) Degradation: the target transcription factor is destabilized by an intein-mediated ligation of an active degron. (v) Spatial separation/localization: the target transcription factor is removed from the nucleus by intein-mediated exchange of the nuclear localization signal with a nuclear export signal. Observe that, unlike the basic antithetic integral motif where the sequestration reaction annihilates the function of the controller molecules Z₁ and Z₂, these intein-mediated reactions may give rise to one or two products that are functional (for example repressors of gene expression). This feature gives rise to more degrees of freedom in the actuation of the regulated network as compared to the standalone antithetic integral motif.

FIG. 2 shows a general framework for intein-based controllers capable of ensuring robust perfect adaptation. The closed-loop network is comprised of the regulated network that is connected with a feedback controller network. The two networks communicate with each other via the input molecule X₁ and output molecule X_{L} of the regulated network. The end goal of the controller network is to ensure Robust Perfect Adaptation (RPA) for the regulated output molecule X_{L}, that is, the controller needs to steer the concentration of the regulated output molecule X_{L} to a prescribed value, called the setpoint, despite uncertainties and constant disturbanced in the regulated network. This is done by sensing the levels of the regulated output molecule X_{L} and accordingly applying a feedback actuation by producing and/or removing the input molecule X₁ in a smart autonomous fashion. The setpoint and sensing mechanisms are embedded in the intein production reactions, more precisely in μ₁, μ₂, f₁ and f₂. Note that in all the figures, lines with arrowheads indicate activation, lines with T-shaped heads indicate inhibition, and lines with diamond shaped heads can indicate both.

FIG. 3 shows the description of the regulated network. The regulated network here is a simple transcription process where an mRNA molecule, denoted by X, is the regulated output. For the closed-loop circuit, the mRNA molecule is transcribed from a gene that encodes for IntN and a fluorescent protein mVenus separated by a linker. The mRNA is then translated into two separate proteins. The first one is the IntN protein which acts as a sensor capable of driving the feedback controller circuit. The second one is the mVenus fluorescent protein which acts as a proxy reporter enabling the inventors to collect fluorescence measurements. For the open-loop circuit, IntN is replaced with IntC or unpaired IntN which cannot drive the controller feedback circuit and thus breaks the loop. To test for RPA, a disturbance is applied by increasing the plasmid copy numbers via transient transfection.

FIG. 4 shows ZF Circuit. (a) Schematic demonstration of the closed- and open-loop circuits implementing a PI controller using a ZF-based transcription factor. Plasmid 1 here expresses a transcription factor which is comprised of a DBD and an AD separated by an IntC and acts as an activator that initiates the transcription of the mRNA X. When the intein subunits (in molecules Z₁ and Z₂) undergo the intein-splicing reaction, they essentially remove the AD and produce Z₃ which is comprised of the DBD without the AD and, as a result, acts as a repressor. The setpoint is tuned by the copy number of the Plasmid 1: more plasmids lead to higher fluorescence levels. The classification of the various molecules and reactions are shown in the box and the hexagons, respectively. (b) Experimental Demonstration of RPA. The two bar graphs depict the normalized fluorescent levels (in %) of the reporter at steady-state, acting as a proxy for the regulated output X, over a wide range of controller plasmid copy numbers (in pg and ng) that dictate the setpoint. Green bars denote the undisturbed (reference) fluorescence levels; whereas, red bars denote the fluorescence levels after a disturbance is applied by increasing the copy numbers of Plasmid 2. The left bar graph corresponds to the response of the closed-loop circuit while the right one corresponds to the open-loop circuit. Clearly, the closed-loop circuit is capable of rejecting the disturbances over a broad range of setpoints to yield an average error (across all setpoints) of 1.7%, while the open-loop circuit fails to do so.

FIG. 5 shows tetR Circuit. (a) Schematic demonstration of the closed- and open-loop circuits implementing an I controller using a tetR-based transcription factor. Plasmid 1 here expresses a transcription factor which is comprised of a DBD, an AD and a DD broken into two pieces that are separated by an IntC. The expressed transcription factor has to dimerize before it can act as an activator that initiates the transcription of the mRNA X. When the intein subunits in molecules Z₁ and Z₂) undergo an intein-splicing reaction, they essentially separate the two pieces of the DD to yield a complex Z₃ that has no function. Furthermore, the dimer Z₄, having two IntC segments, can also undergo an intein-splicing reaction to give back the monomer Z₁ and broken complex Z₃. The setpoint is tuned by the copy number of Plasmid 1: more controller plasmids lead to higher fluorescence levels. The classification of the various molecules and reactions are shown in the box and the hexagons, respectively. (b) Experimental Demonstration of RPA. The two bar graphs depict the normalized fluorescent levels (in %) of the reporter at steady-state, acting as a proxy for the regulated output X, over a wide range of Plasmid 1 copy numbers (in pg and ng) that dictate the setpoint. Green bars denote the undisturbed (reference) fluorescence levels; whereas, red bars denote the fluorescence levels after a disturbance is applied by increasing the copy numbers of Plasmid 2. The left bar graph corresponds to the response of the closed-loop circuit while the right one corresponds to the open-loop circuit. Clearly, the closed-loop circuit is capable of rejecting the disturbances over a broad range of setpoints to yield an average error (across all setpoints) of 5.7%, while the open-loop circuit fails to do so.

FIG. 6 shows tTA Circuit. (a) Schematic demonstration of the closed- and open-loop circuits implementing a PI controller using a tTA-based transcription factor. Plasmid 1 here expresses a transcription factor which is comprised of a DBD, DD, IntC and AD. The expressed transcription factor has to dimerize before it can act as an activator that initiates the transcription of the mRNA X. When the intein subunits in molecules Z₁ and Z₂) undergo an intein-splicing reaction, they essentially essentially remove the AD. Without the AD, Z₃ still has a DD and as a result can still dimerize to produce the dimer Z₄ that acts as a repressor since it lacks an AD. Note that Z₁ can either homo-dimerize or hetero-dimerize with Z₃ to yield Z₅ and Z₆ that can act as activators since they have an AD. However, Z₅ is expected to be a stronger activator than Z6 since it has an additional AD. Finally, both dimers Z₅ and Z₆ have active IntC segments and, as a result, can also undergo intein-splicing reactions with Z₂. The setpoint is tuned by the copy number of Plasmid 1: more controller plasmids lead to higher fluorescence levels. The classification of the various molecules and reactions are shown in the box and the hexagons, respectively. (b) Experimental Demonstration of RPA. The two bar graphs depict the normalized fluorescent levels (in %) of the reporter at steady-state, acting as a proxy for the regulated output X, over a wide range of Plasmid 1 copy numbers (in pg and ng) that dictate the setpoint. Green bars denote the undisturbed (reference) fluorescence levels; whereas, red bars denote the fluorescence levels after a disturbance is applied by increasing the copy numbers of Plasmid 2. The left bar graph corresponds to the response of the closed-loop circuit while the right one corresponds to the open-loop circuit. Clearly, the closed-loop circuit is capable of rejecting the disturbances over a broad range of setpoints to yield an average error (across all setpoints) of −4.1%, while the open-loop circuit fails to do so.

FIG. 7 shows GAL4 Circuit. (a) Schematic demonstration of the closed- and open-loop circuits implementing a PI controller using a GAL4-based transcription factor. Plasmid 1 here expresses a transcription factor whose domains are the same as those in the tTA circuit of FIG. 6(a), but with only one difference: the order of IntC and the DD is interchanged. This leads to the exact same network topology as that of the tTA circuit of FIG. 6(a), however the molecules Z₃, Z₄ and Z₆ lack a DBD and thus are incapable of binding to the DNA. As a result, the dimer Z₅ is the only activator here, and there are no direct repressors. The setpoint is tuned by the copy number of Plasmid 1: more controller plasmids lead to higher fluorescence levels. The classification of the various molecules and reactions are shown in the box and the hexagons, respectively. (b) Experimental Demonstration of RPA. The two bar graphs depict the normalized fluorescent levels (in %) of the reporter at steady-state, acting as a proxy for the regulated output X, over a wide range of Plasmid 1 copy numbers (in pg and ng) that dictate the setpoint. Green bars denote the undisturbed (reference) fluorescence levels; whereas, red bars denote the fluorescence levels after a disturbance is applied by increasing the copy numbers of Plasmid 2. The left bar graph corresponds to the response of the closed-loop circuit while the right one corresponds to the open-loop circuit. Clearly, the closed-loop circuit is capable of rejecting the disturbances over a broad range of setpoints to yield an average error (across all setpoints) of 0.1%, while the open-loop circuit fails to do so.

FIG. 8 shows TetR-Inactive IntN Circuit. (a) Schematic demonstration of the closed- and open-loop circuits implementing an I controller using a transcription factor complex. Plasmid 1 here expresses a fusion protein comprised of IntC and AD. Plasmid 2 here expresses a fusion protein comprised of an inactive IntN and tetR. Two fusion proteins can reversibly heterodimerize through the association between the inactive IntN and IntC and form a functional transcription factor, which acts as an activator that initiates the transcription of the mRNA X. When the intein subunits (in molecules Z₁ and Z₂) undergo the intein-sequestration reaction, they essentially remove the InC from AD in molecule Z1. As a result, AD no longer dimerizes with the DNA binding domain. The setpoint is tuned by the copy number of the Plasmid 1: more plasmids lead to higher fluorescence levels. The classification of the various molecules and reactions are shown in the box and the hexagons, respectively. (b) Experimental Demonstration of RPA. The two bar graphs depict the normalized fluorescent levels (in %) of the reporter at steady-state, acting as a proxy for the regulated output X, over a wide range of controller plasmid copy numbers (in pg and ng) that dictate the setpoint. Green bars denote the undisturbed (reference) fluorescence levels; whereas, red bars denote the fluorescence levels after a disturbance is applied by increasing the copy numbers of Plasmid 2. The left bar graph corresponds to the response of the closed-loop circuit while the right one corresponds to the open-loop circuit. Clearly, the closed-loop circuit is capable of rejecting the disturbances over a broad range of setpoints to yield an average error (across all setpoints) of 3.9%, while the open-loop circuit fails to do so.

FIG. 9 shows a topology of multiple complexes.

FIG. 10 shows a first topology of multiple Inteins.

FIG. 11 shows a second topology of multiple Inteins.

FIG. 12 shows a topology of a positive complex.

FIG. 13 shows a topology of a protease.

FIG. 14 shows a clean version of the regulatory network.

FIG. 15 Intein-based controller for cytokine regulation. (a) A schematic diagram of the co-culture assay designed to evaluate the performance of the TNF-α controller. (b) Synthetic circuits stably integrated into different cell types for unique functions (c) Temporal dynamics of TNF-α levels as measured by reporter cells, demonstrating the controller's disturbance rejection capabilities. (d) Mean fluorescence intensity derived from reporter cells' TNF-α reporter and controller cells' set-point marker, demonstrating the controller's set-point tracking ability. (e) A schematic representation highlighting the potential application of intein-based circuits as safety mechanisms in CAR-T cell therapy.

FIG. 16 Characterizing the dynamic response of intein-based controllers with a hybrid in silico/in vivo platform. (a) The Cyberloop framework for studying the dynamic performance of biomolecular controllers. (b) The genetic architecture of an intein-based controller for Cyberloop interfacing (e.g. in silico one-species plant). (c) Closed-loop dynamics of a single controller clone with a birth-death process plant, demonstrating disturbance rejection. (d) Closed-loop dynamics of a single controller clone (blue) with a type 1 diabetes disease model. Note that the set-point can be tuned to be in the desired range. Simulations of different in silico patients (red), without glucose control.

FIG. 17 Engineering intein-based bacterial circuits for robust perfect adaptation.

FIG. 18 Steady-state and dynamic behavior of intein-based bacterial circuits. a) Top: steady-state measurements of four different open-loop circuits with different fixed output setpoints with and without an aTc disturbance. Bottom: Time course of one open-loop strain with an aTc disturbance applied at 1 hour. b) Top: steady-state measurements of one proportional-integral circuit. Output setpoints are varied by inducing cells with different concentrations of arabinose. Bottom: Time course of the proportional-integral circuit induced with 0.5% arabinose and an aTc disturbance applied at 1 hour. c) Top: steady-state measurements of two proportional circuits. Output setpoints are varied by inducing cells with different concentrations of arabinose. Bottom: Time course of proportional circuits induced with 0.5% arabinose and an aTc disturbance applied at 1 hour. Data show mean±s.d. for n=2 biological replicates.

FIG. 19 Comparison of present invention to (Muldoon et al. Science Advances (2021). 7(8), p. 1085-1090) and WO 2022/060927 A1.

FIG. 20 The figure shows three different possible ways in which the production rate of the anti-controller can depend on y. Each will give rise to a different setpoint for the same production rate of the controller (p). So by changing the way f(y) depends on y (extent and manner of dependence on y), one can get different setpoints y1*, y2*, or y3*. For a given dependency (f(y)), one can alternatively change the stepping by adjusting the production rate of the controller (p). That has the effect of moving the horizontal line up or down, which changes the intersection point.

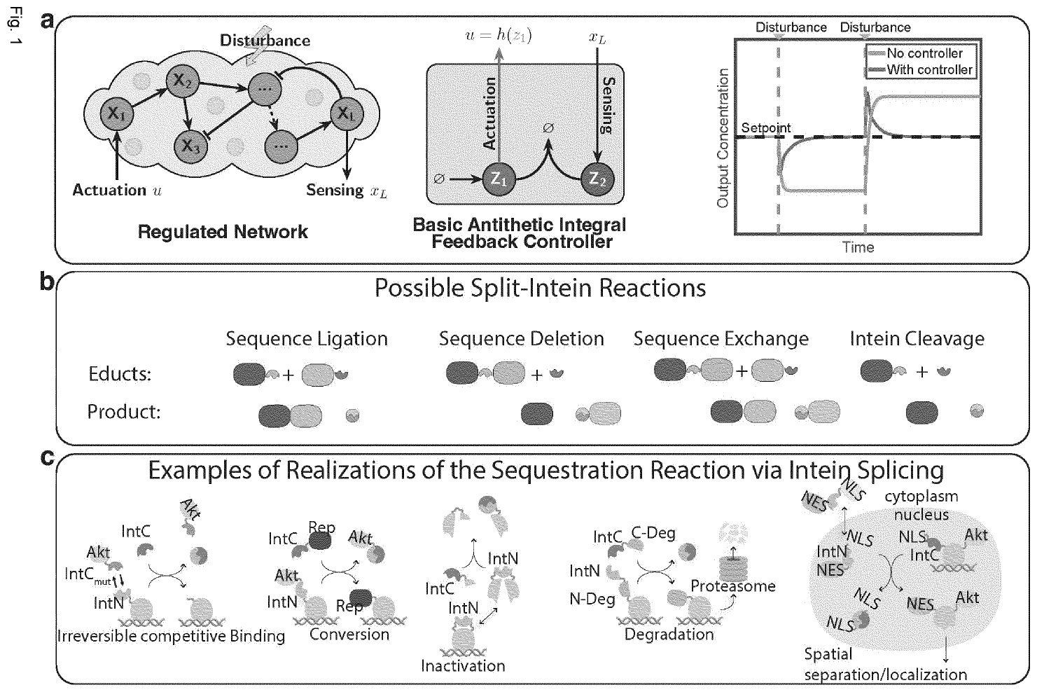

### EXAMPLES

### Example 1: General Topologies for Intein-Based Integral Controllers

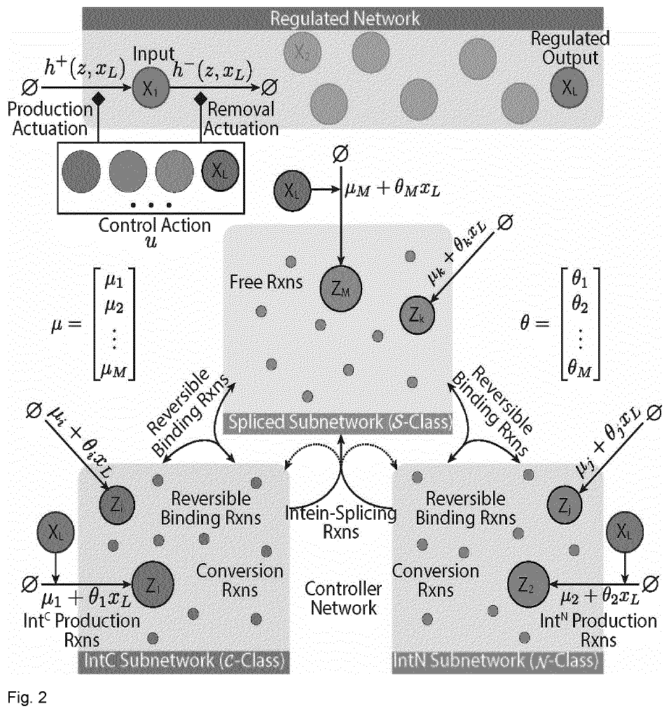

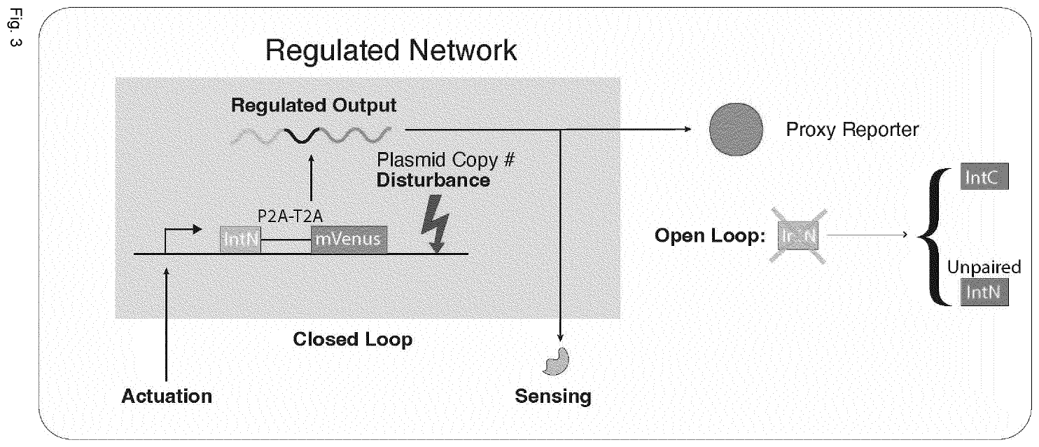

In this section, the inventors describe a broad class of network topologies that are capable of building intein-based integral controllers that ensure RPA. Consider the general closed-loop network depicted in FIG. 2 where an arbitrary network comprised of L species X:={X₁, . . . , X_{L}}, referred to as the regulated network, is in a feedback interconnection with the controller network comprised of M species Z:={Z₁, . . . , Z_{M}}.

The overall objective of the feedback controller is to achieve RPA for the regulated output molecule X_{L} by actuating the input molecule X₁. The controller network is divided into three subnetworks that belong to one of three classes: the N-class, the C-class and the S-class. Controller molecules are classified according to the following rules:

### Molecule Rules:

1. All controller molecules containing at least one active IntC belong to the C-class.
2. All controller molecules containing at least one active IntN belong to the N-class.
3. All controller molecules that do not contain any active inteins belong to the S-class.

The various molecules in the three classes react according to the following rules:

### Reaction Rules:

1. Productions of controller molecules are either constitutive or driven by the regulated output molecule XL
2. Any controller molecule in the C-class can undergo an intein-splicing reaction with any controller molecule in the N-class to produce at least one product, one of which belongs to the S-class.
3. The input molecule X₁ is either produced or degraded or both by at least one of the controller molecules.
4. Within each class, controller molecules are allowed to convert to one another while preserving the number of IntC/IntN.
5. Within each class, controller molecules are allowed to reversibly bind to one another while preserving the number of IntC/IntN.
6. Controller molecules belonging to the C-class are allowed to reversibly bind with controller molecules belonging to the S-class to produce another controller molecules belonging to the C-class while preserving the number of IntC/IntN.
7. Controller molecules belonging to the S-class are allowed to be produced, degraded, and freely react among
8. Controller molecules belonging to the S-class are allowed to be produced, degraded, and freely react among each other to yield controller molecules that remain within the S-class.

### Example 2: Intein-Based Genetic PI Controllers

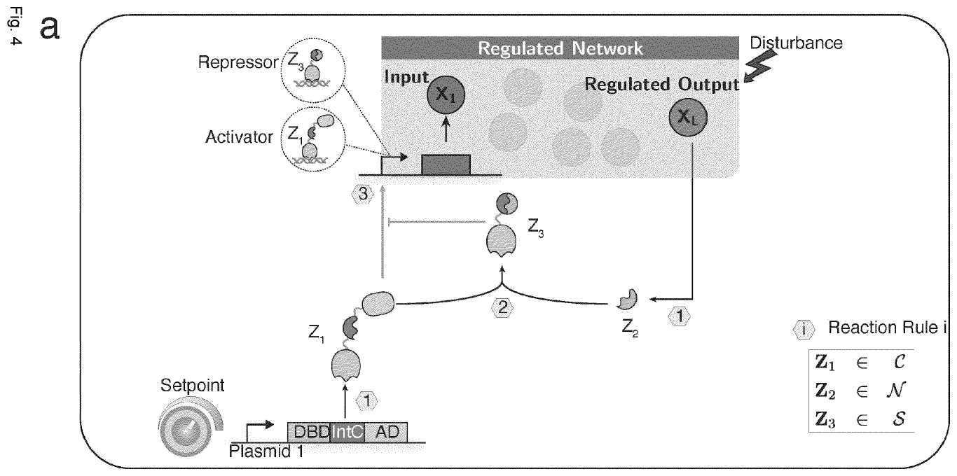

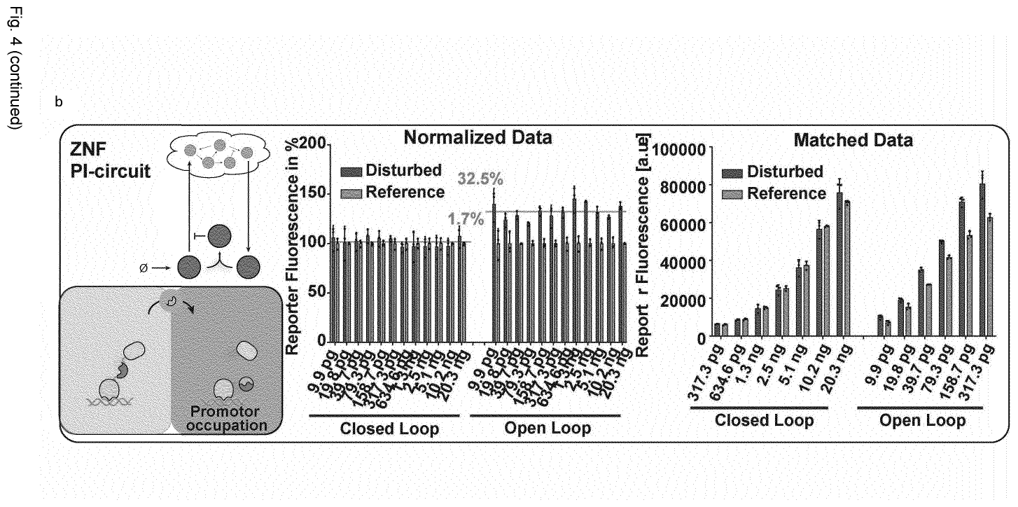

In this section, the inventors present several intein-based controller circuits that are specific embodiments of the general topology presented in the previous section. These embodiments depend on the specific transcription factors used and the insertion location of the intein as well. The inventors begin with the simplest controller circuit and then move on to a higher level of complexity. To experimentally demonstrate that all the presented controllers achieve RPA, the inventors use the same regulated network depicted in FIG. 3 as an example. The objective here is to drive the concentration levels of the regulated output X (measured via the proxy reporter) to a prescribed setpoint, despite the disturbance which is applied here by increasing the plasmid copy numbers as illustrated in FIG. 3.

This RPA property is tested for a wide range of setpoints for all of the controllers presented next. In what follows, let DBD, DD and AD denote the DNA binding domain, dimerization domain and activation domain, respectively.

### Zinc Finger (ZF) Circuit

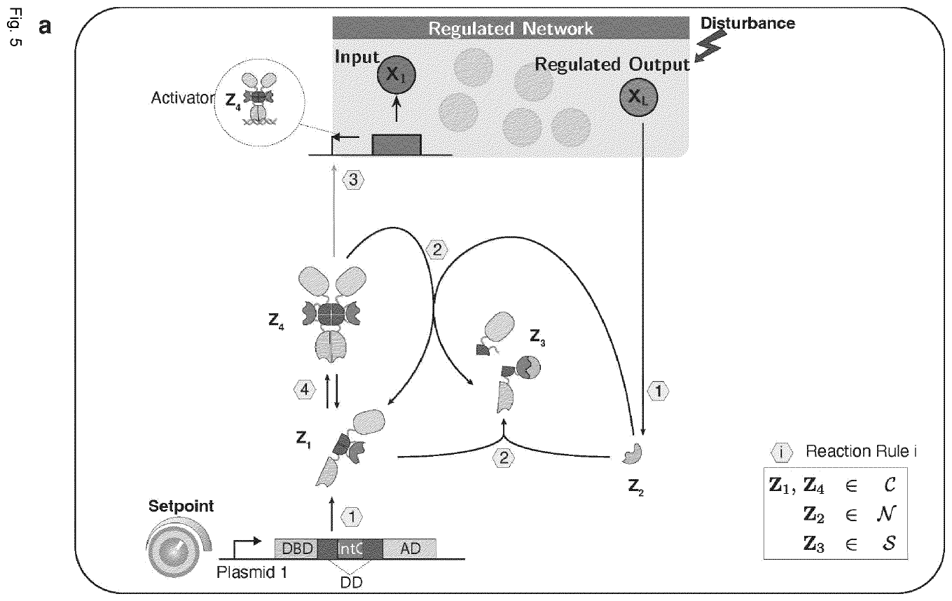

In this controller circuit, the used transcription factor is based on ZF and is monomeric. That is, it is capable of activating transcription as a monomer (no dimerization reactions). The closed- and open-loop circuits are schematically depicted in FIG. 4(a) where the classification of the various controller molecules and the reaction rules are also shown. The activation by molecule Z₁ gives rise to an integral controller, while the repression by molecule Z₃ gives rise to a (filtered) proportional controller and thus summing up to a PI controller. The responses of the circuits depicted in FIG. 4(b) demonstrate that the closed-loop circuit achieves RPA over a broad range of setpoints, while the open-loop circuit fails to do so.

tetR Circuit

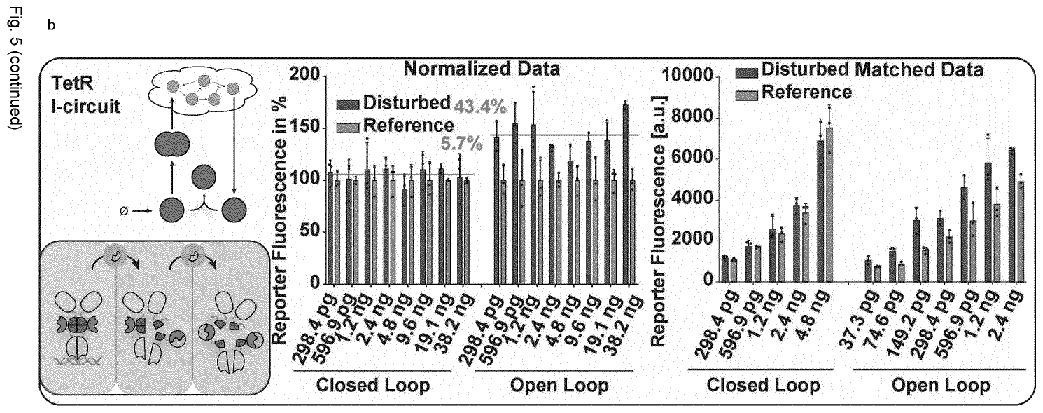

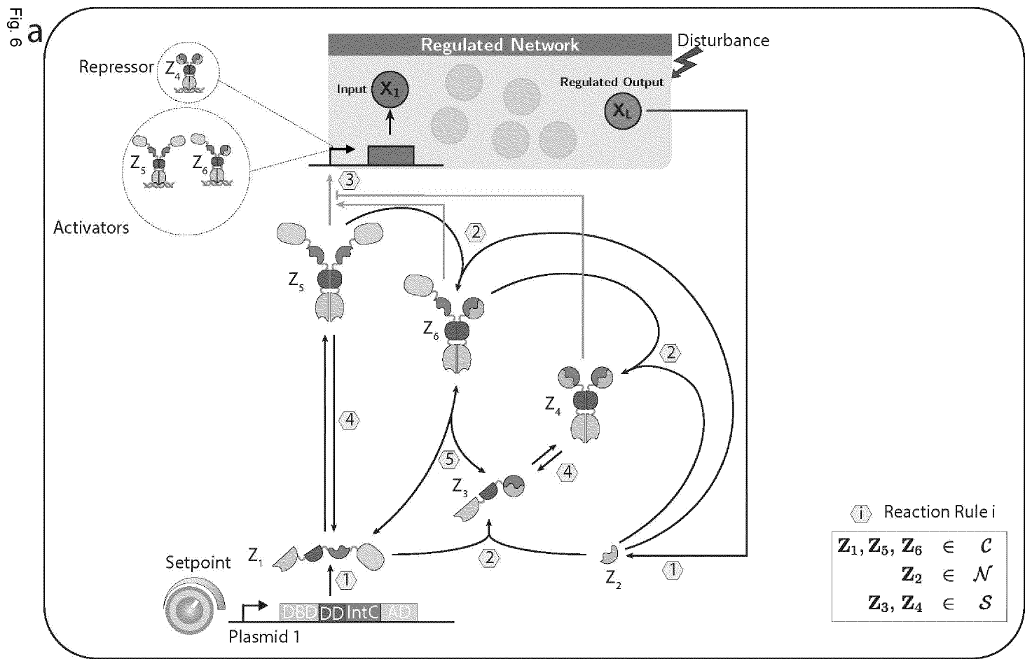

In this controller circuit, the used transcription factor is based on tetR and is dimeric. That is, it is only capable of activating transcription as a dimer. The closed- and open-loop circuits are schematically depicted in FIG. 5(a) where the classification of the various controller molecules and the reaction rules are also shown. The activation by molecule Z₁ gives rise to an integral controller. The responses of the circuits depicted in FIG. 5(b) demonstrate that the closed-loop circuit achieves RPA over a broad range of setpoints, while the open-loop circuit fails to do so.

### tTA Circuit

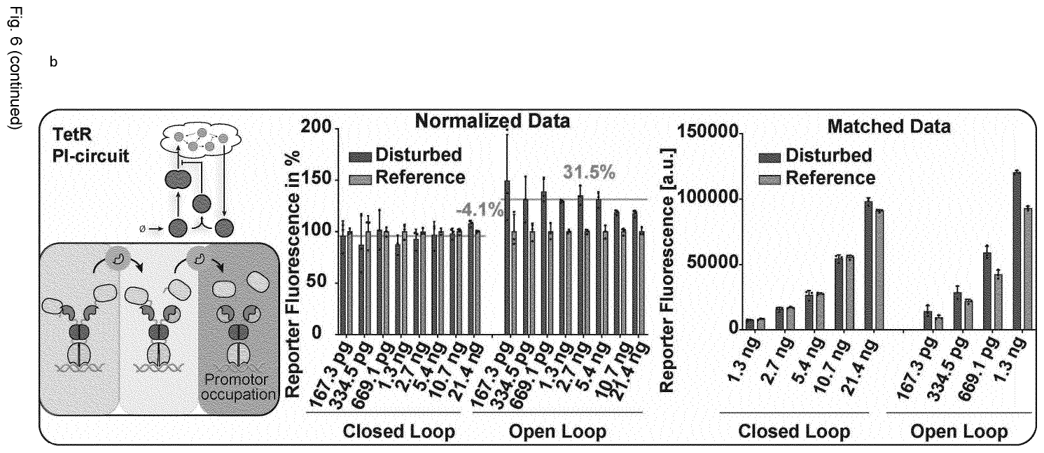

In this controller circuit, the used transcription factor is based on tTA and is dimeric. That is, it is only capable of activating transcription as a dimer. The closed- and open-loop circuits are schematically depicted in FIG. 6(a) where the classification of the various controller molecules and the reaction rules are also shown. The activation by molecules Z₄ and Z₆ give rise to an integral controller, while the repression by molecule Z₄ gives rise to (filtered) proportional controller and thus summing up to a PI controller. The responses of the circuits depicted in FIG. 6(b) demonstrate that the closed-loop circuit achieves RPA over a broad range of setpoints, while the open-loop circuit fails to do so.

### GAL4 Circuit

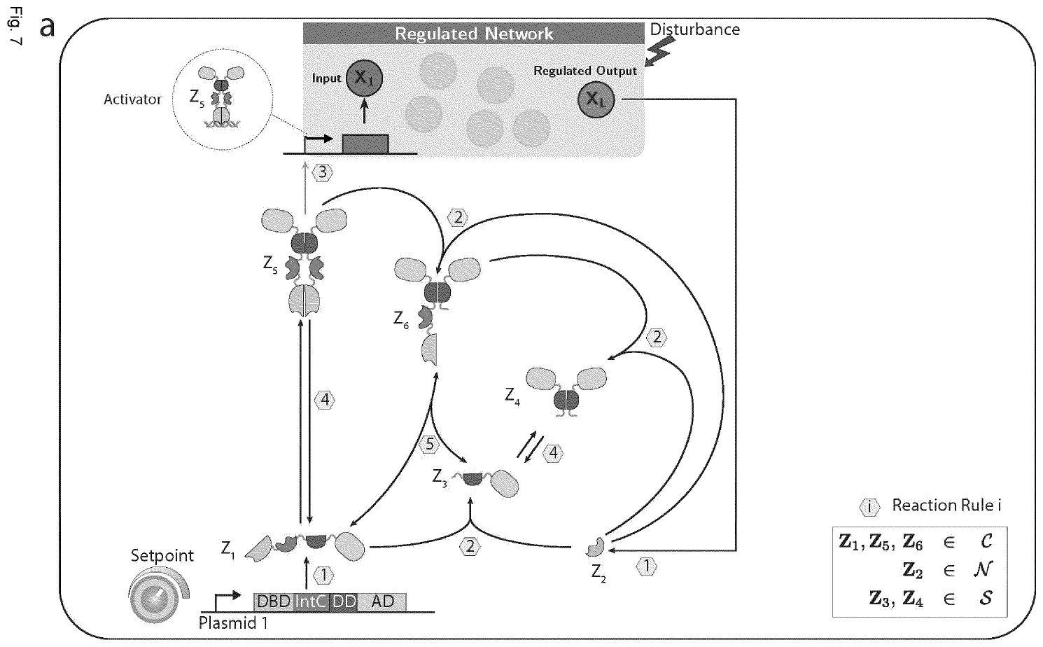

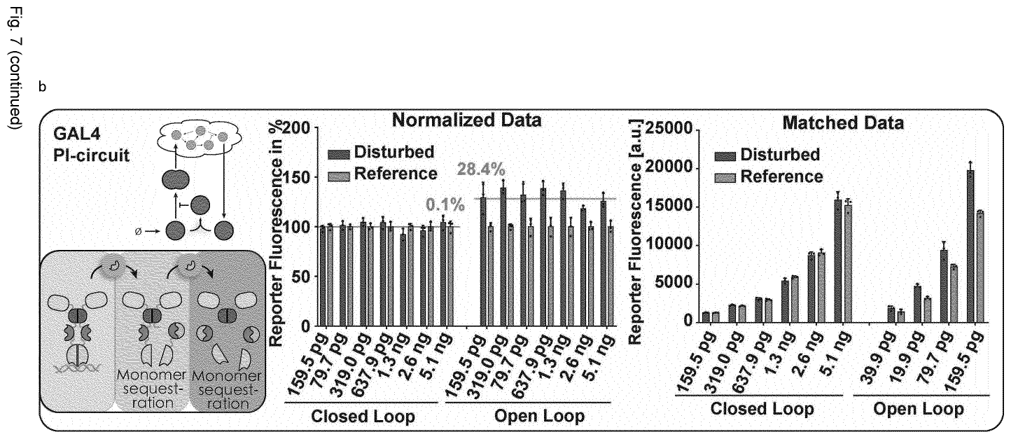

In this controller circuit, the used transcription factor is based on GAL4 and is dimeric. That is, it is only capable of activating transcription as a dimer. The closed- and open-loop circuits are schematically depicted in FIG. 7(a) where the classification of the various controller molecules and the reaction rules are also shown. The activation by molecule Z₅ gives rise to an integral controller, while the intein-splicing of Z₅ by Z₂ acts as a repression mechanism giving rise to a (filtered) proportional controller and thus summing up to a PI controller. The responses of the circuits depicted in FIG. 7(b) demonstrate that the closed-loop circuit achieves RPA over a broad range of setpoints, while the open-loop circuit fails to do so.

tetR-Inactive IntN Circuit

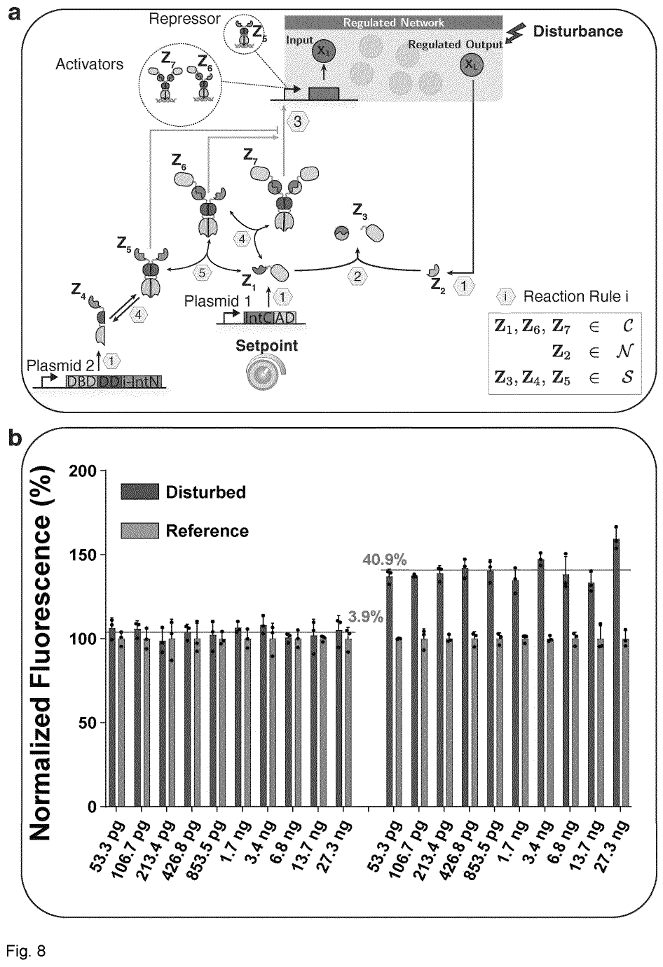

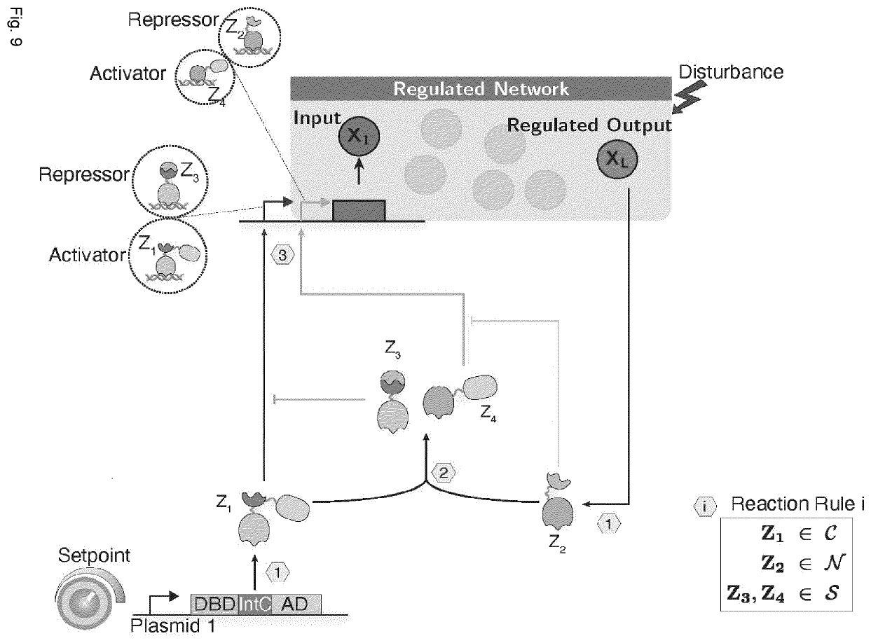

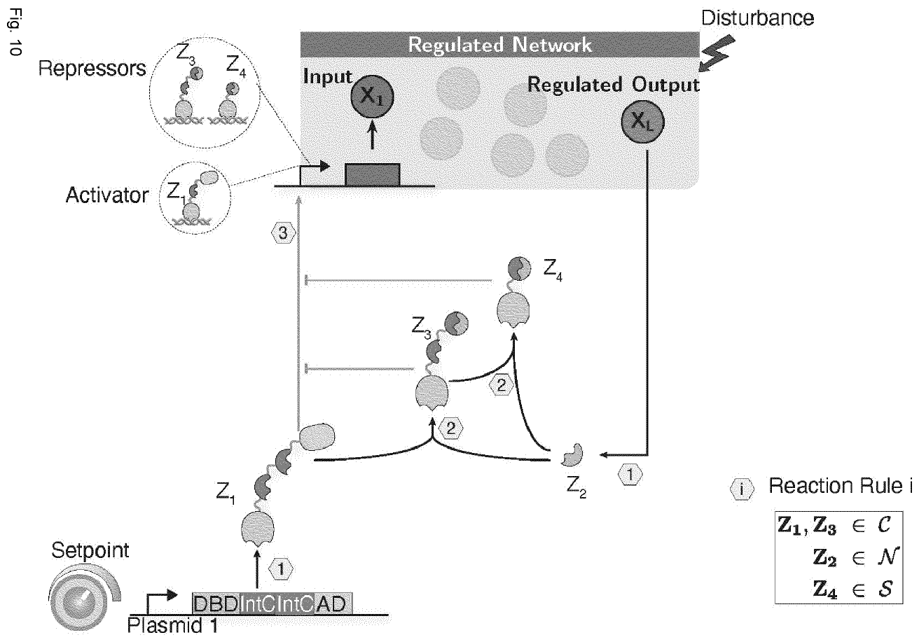

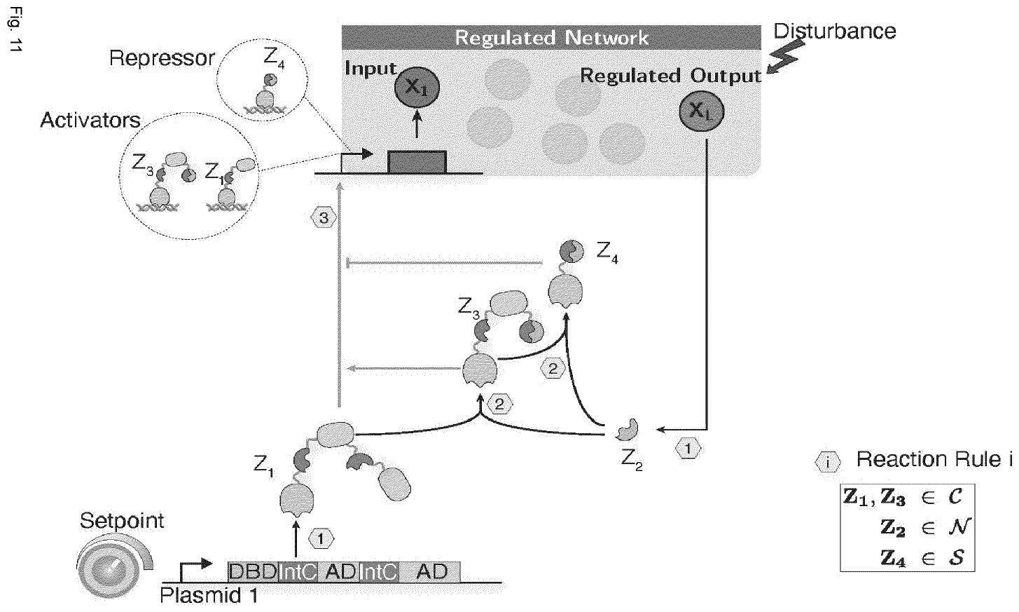

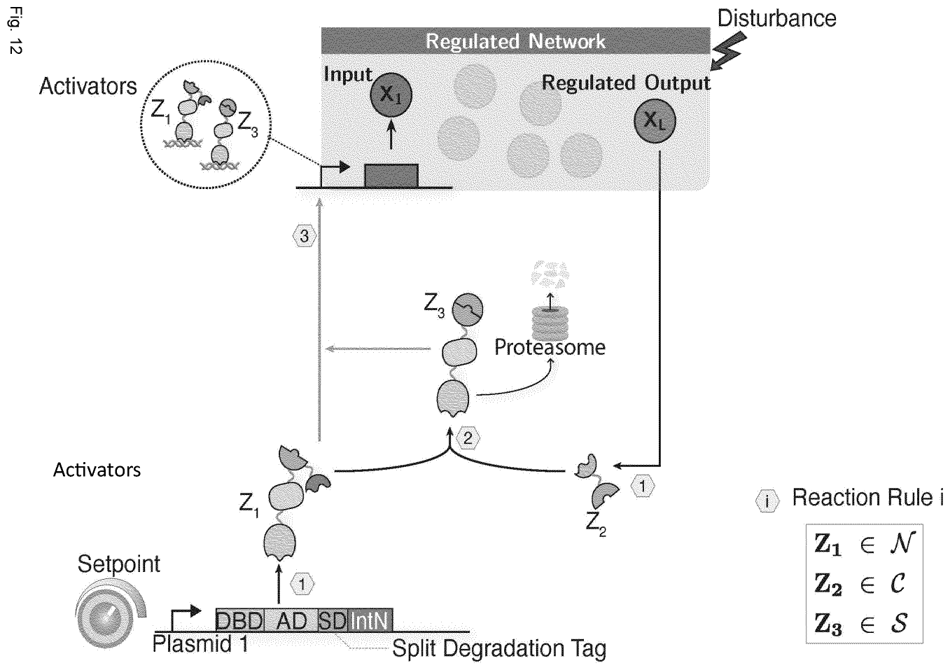

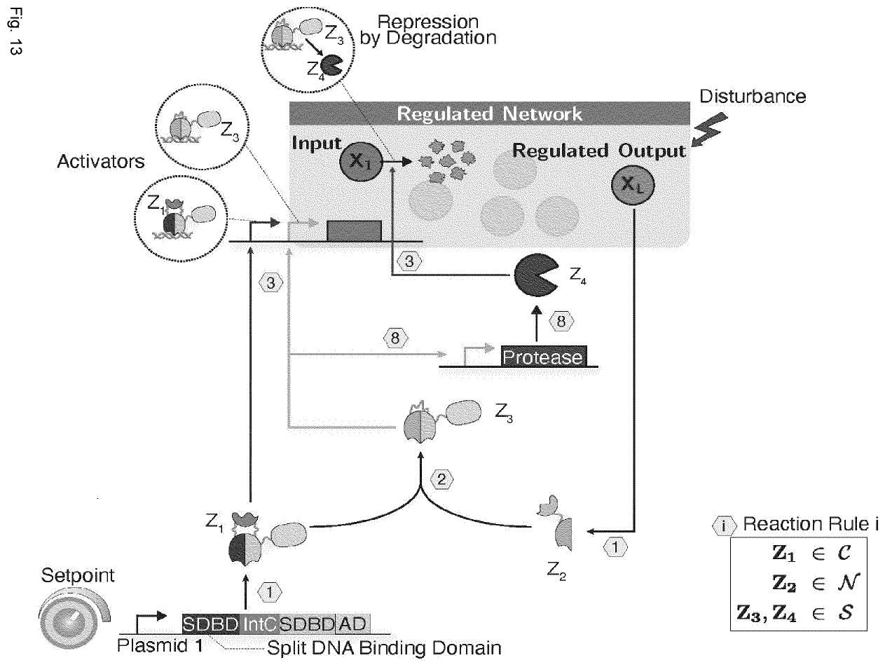

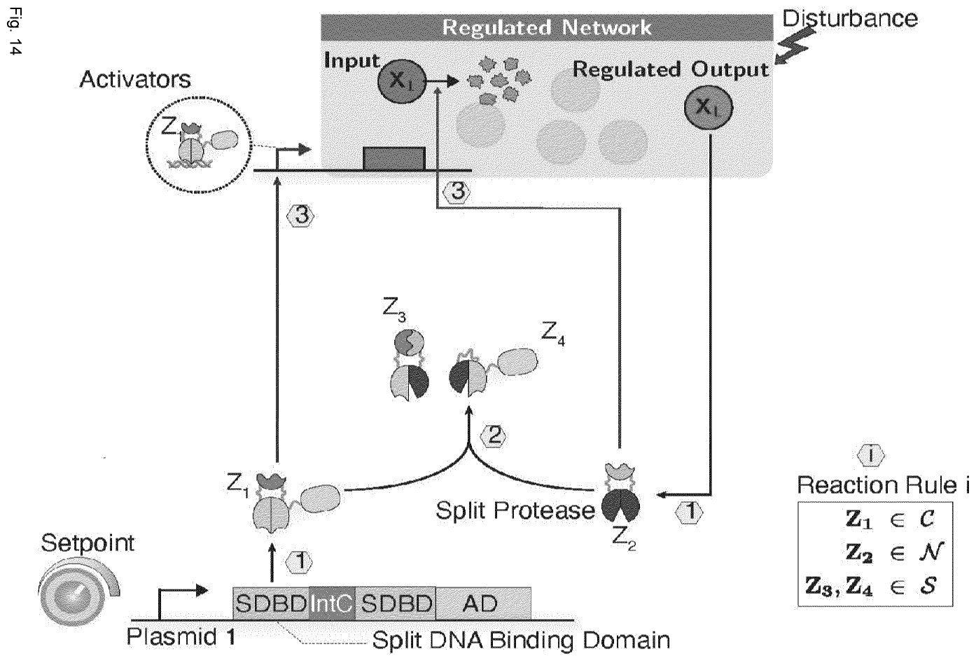

In this controller circuit, the used transcription factor is based on complex formation between two fusion proteins: inactive IntN fused with a tetR DNA-binding domain and IntC fused with activation domain. That is, it is only capable of activating transcription as a complex. The closed- and open-loop circuits are schematically depicted in FIG. 8(a) where the classification of the various controller molecules and the reaction rules are also shown. The activation by controller molecules Z₆ and Z₇ gives rise to an integral controller. The responses of the circuits depicted in FIG. 8(b) demonstrate that the closed-loop circuit achieves RPA over a broad range of setpoints, while the open-loop circuit fails to do so.

### Example 3: Engineering Intein-Based Controllers for Cytokine Regulation

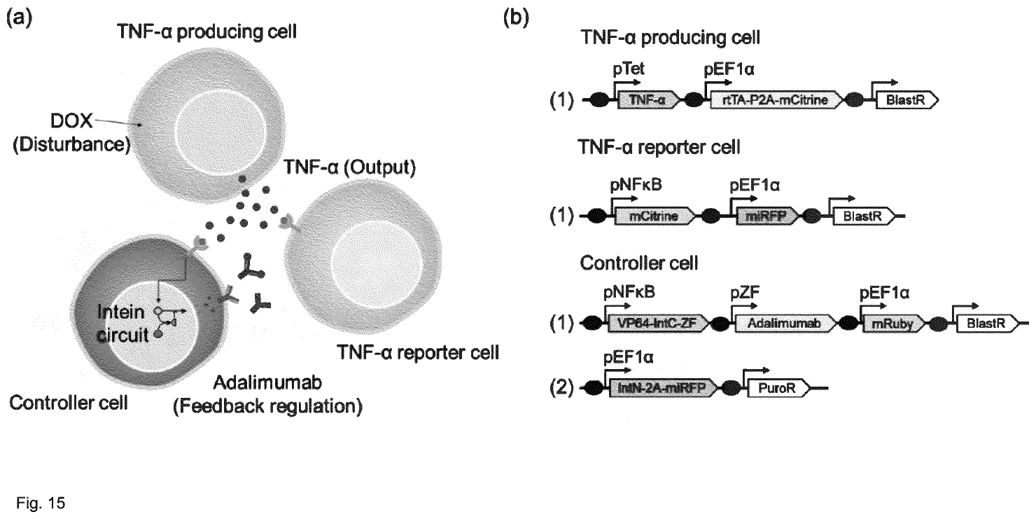

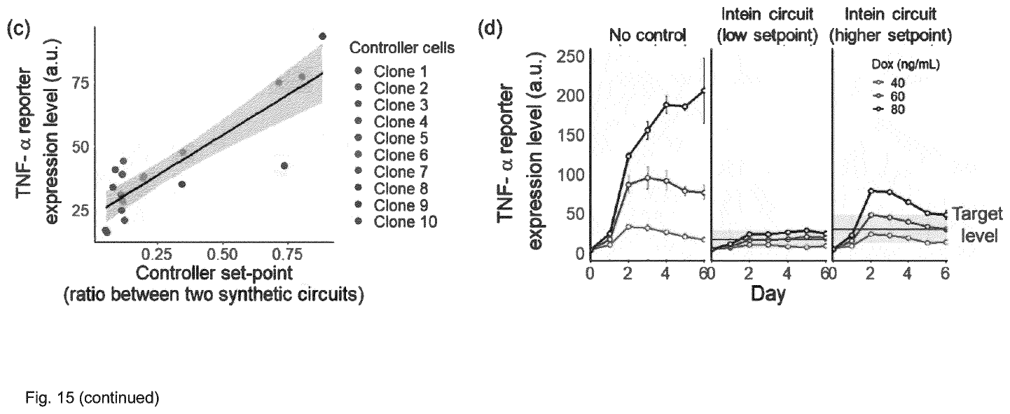

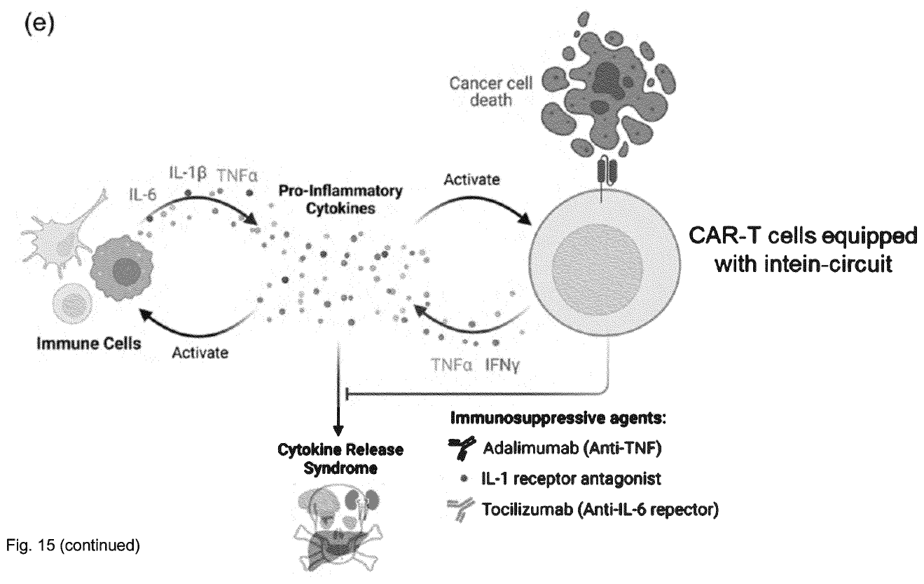

TNF-α, a crucial pro-inflammatory cytokine, plays a significant role in various immune processes. Its elevated concentration in the body is associated with several diseases, ranging from chronic inflammation to acute cytokine release syndrome. While TNF-α inhibitors have been developed and shown to be effective in preventing an overreactive immune system, they often lead to significant immunosuppression, resulting in increased susceptibility to infections. As such, maintaining TNF-α within specific concentrations is critical. To meet this challenge, we employed an intein-based controller to regulate TNF-α levels. Cells engineered with this controller have the capability to continuously monitor TNF-α concentrations and supply appropriate levels of the TNF-α inhibitor, Adalimumab. The two key functionalities of the controller cells, namely set-point tracking and disturbance rejection, were evaluated through in vitro co-culture time course experiment (FIG. 15a). The experiment involved three cell types, each incorporated with stably integrated synthetic circuits for unique functions: 1. Controller cells equipped with the intein controller; 2. TNF-α producing cells, capable of generating TNF-α under DOX induction; 3. TNF-α reporter cells, designed to provide real-time measurement of TNF-α activity throughout the experiment (FIG. 15b). The set-point of intein controller is determined by the ratio between two synthetic circuits—output sensing circuit and reference encoded circuit. This ratio was measured by the disparity in expression levels of the fluorescent reporter encoded within the two synthetic circuits. After six days of co-culture, the TNF-α level measured by the reporter cells was positively correlated with the controller's set-point level (FIG. 15c). As shown in FIG. 15d, without the controller present in the co-culture, DOX-induced disturbance significantly escalates TNF-α concentration. Nevertheless, cells with the intein-based controller can manage TNF-α at a specific level, unaffected by the varied TNF-α production rates induced by DOX. These results demonstrated the potential of the intein-based controller for TNF-α regulation. In a more clinically relevant example of the intein circuits, we highlight the usage of Chimeric Antigen Receptor (CAR) T-cells (FIG. 15e). CAR T-cells have demonstrated significant efficacy in the treatment of blood cancers, but they come with a potentially lethal side effect—Cytokine Release Syndrome (CRS). This syndrome is triggered by the uncontrolled massive release of cytokines, resulting from the interaction between engineered cells and the patient's innate immune system. Although various immunosuppressive agents, such as the anti-IL6 receptor (Tocilizumab), anti-TNF (Adalimumab), and IL-1 receptor antagonist, can mitigate CRS, their use requires constant patient monitoring to ensure the right dosage is administered at the right time. The intein-based circuit provides an optimal solution to this challenge. By integrating the intein circuit into CAR T-cells, we can equip these cells with a self-guiding system. This mechanism enables the engineered cells to maintain cytokine levels within a safe range to prevent CRS while preserving a robust immune response for the eradication of cancer cells.

### Example 4: Evaluating Intein-Based Controllers for Biomedical Applications with a Cyberloop Framework

The flexibility of inteins allows the construction of advanced biomolecular control systems with improved dynamic performance (e.g. low overshoot, fast settling time). However, to this date, the mammalian implementations have only studied steady-state behaviors via transient transfection and end-point measurements with flow cytometry. Here, we characterize the dynamic performance of intein-based controllers by interfacing benchmark chemical reaction networks with engineered cells stably expressing the control circuits of interest. To achieve this, we leverage the previously developed Cyberloop (Kumar et al. 2021. Nature Communications 12 (1): 5651.) in silico/in vivo platform as a testbed for mammalian control systems. We also extend this framework to perform testing of candidate therapeutic controllers by employing suitable disease mathematical models. This ‘patient-in-the-loop’ platform can act as a surrogate for animal models and permits high-throughput validation of engineered cells.

When studying closed-loop systems, the composite chemical reaction network can be separated into two networks, the controller and the controlled network (plant). The key principle behind the Cyberloop platform is to physically separate these two networks, by simulating the controlled network inside a computer (in silico) and genetically encoding the controller in cells (in vivo), using microscopy and optogenetics as the interface between the two. This is the reverse setup of what was previously described and reduces experimental complexity, allowing for controller testing without specialized sensor and actuator systems.

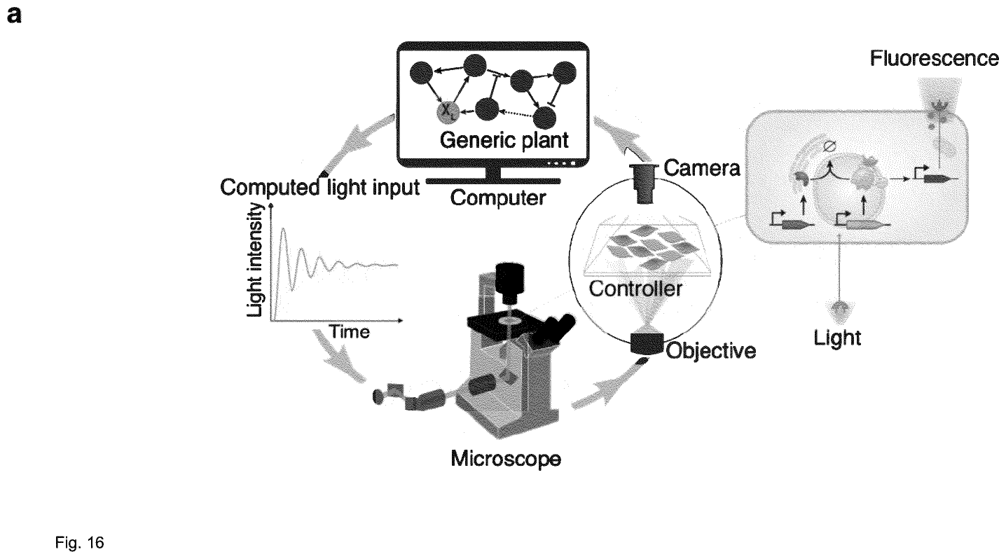

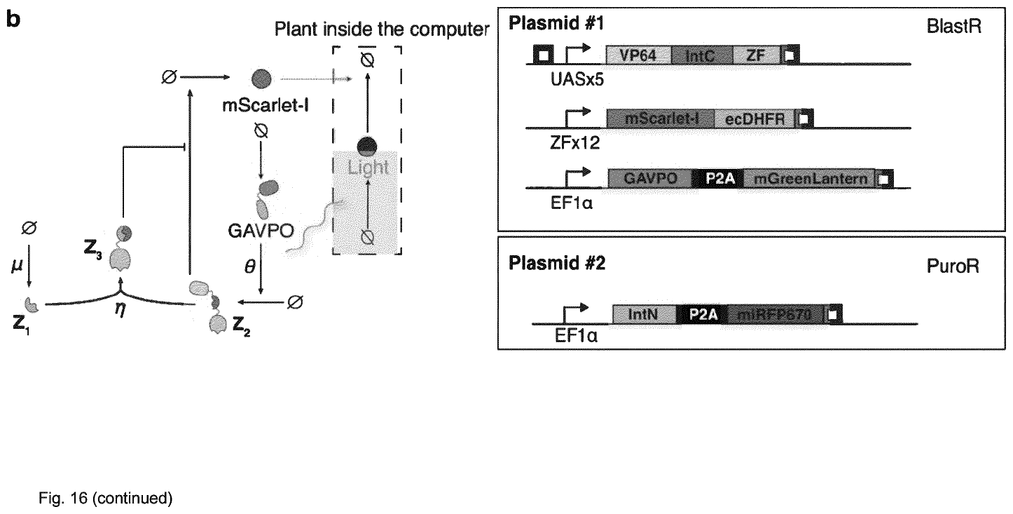

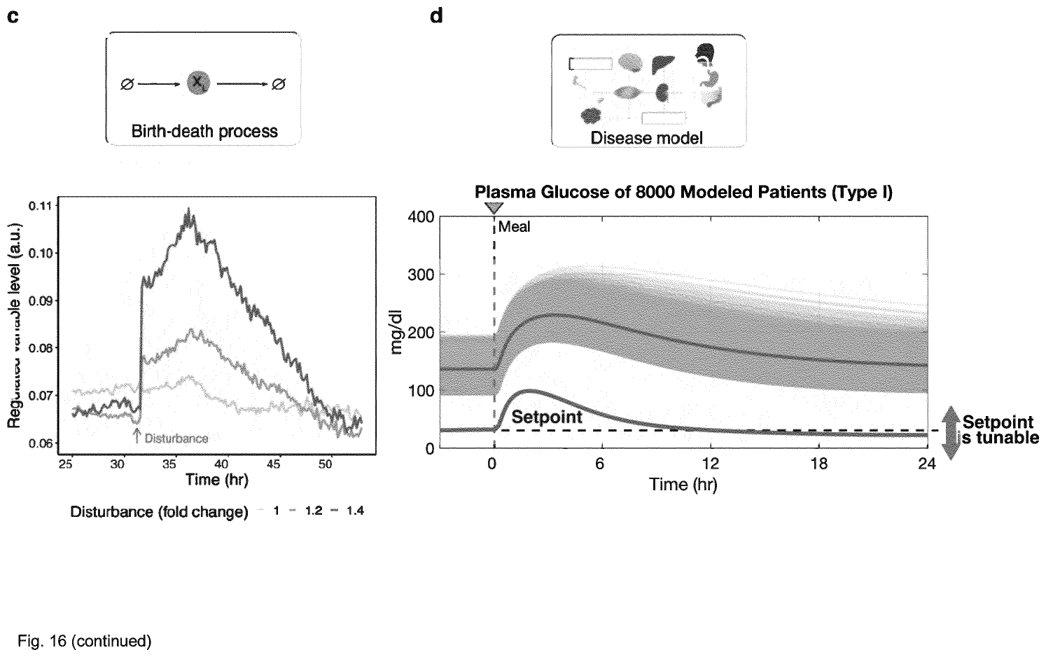

The Cyberloop system requires reliable communication between the in silico and in vivo networks. An inverted epifluorescence microscope is used for this interface, where light (a proxy for the regulated variable) acts via an optogenetic module as the input to the engineered control gene circuit in the cell. The output of the controller is a fluorescent protein, whose abundance is measured during imaging. This readout is used during the simulation of the reaction network to emit the next light input to the candidate controller cells, effectively closing the loop (FIG. 16a). The controlled network (in silico plant) in the computer is a benchmark model of increasing complexity. Preliminary results are provided with an one-species plant (birth-death process) and a FDA-approved Type 1 diabetes model in FIG. 16c and FIG. 16d respectively. The biological module (in vivo controller) is constructed in HEK-293T cells, using an intein-based architecture (FIG. 16b). For the interface, cells are engineered to be light responsive, using the blue light-triggered optogenetic GAVPO/LightON system (Wang et al 2012. Nature Methods 9 (3): 266-69.). The dynamic fluorescent readout needs to have a high signal-to-noise ratio and be fast enough to capture the dynamics of the system. Therefore, a bright fluorescent protein (mScarlet-1) is used, fused with a degradation tag (ecDHFR) to allow for high destabilization and fast time to steady-state.

The integrated platform allows for the interrogation of the dynamic behavior of intein-based controllers. In the case of a simple birth-death process plant, the cell-based controller was able to reject various computational disturbances (increasing the birth rate of the plant species), achieving perfect adaptation and fast settling time (˜1 day) (FIG. 16c). Additionally, the controller was interfaced with a computational model of Type 1 diabetes demonstrating postprandial glucose regulation after a meal simulation with 45 g of glucose (FIG. 16d).

### Example 5: Engineering Intein-Based Controllers in Bacteria

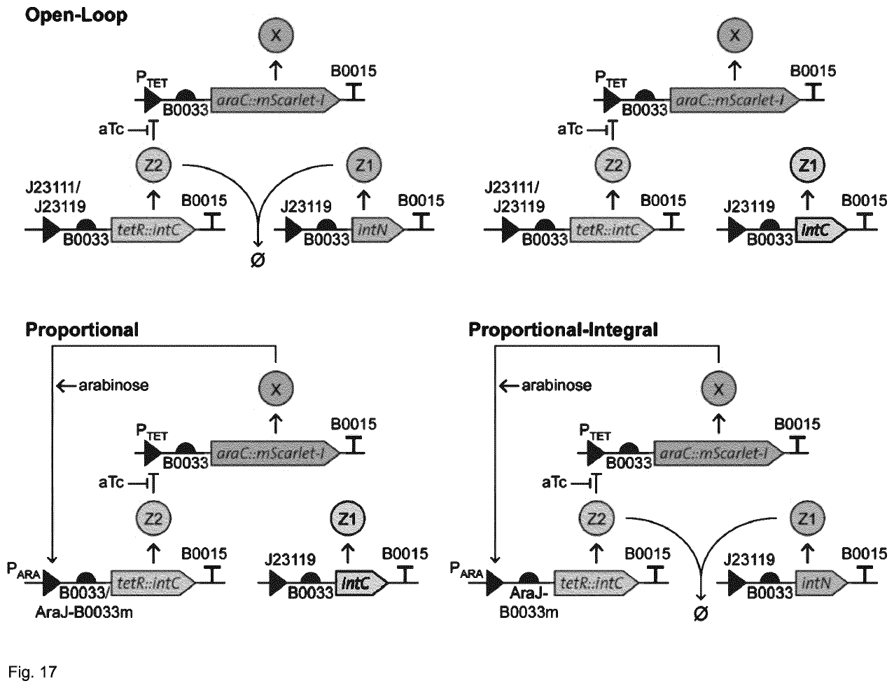

The previous points demonstrate the usage of intein-based controllers in mammalian cells. To demonstrate that intein-based integral controllers can be extended to other organisms, the GP41-1 TetR-IntC and IntN sequestration pair described under “Example 2: Intein-Based Genetic PI Controllers” in the tetR Circuit section was transferred to Escherichia coli for the purpose of constructing a PI controller in bacteria (FIG. 17). TetR-IntC, in this case, represses the transcription of the fluorescently-tagged transcription factor output V5::araC::mScarlet-I. V5-AraC-mScarlet-I, in turn, activates transcription of tetR::intC. IntN, which is constitutively produced, annihilates TetR-IntC by cleaving the TetR dimerization domain, preventing TetR dimerization and binding of the repressor to its cognate promoter. Actuating with the output sensing controller species inherently adds a proportional component to the integral feedback.

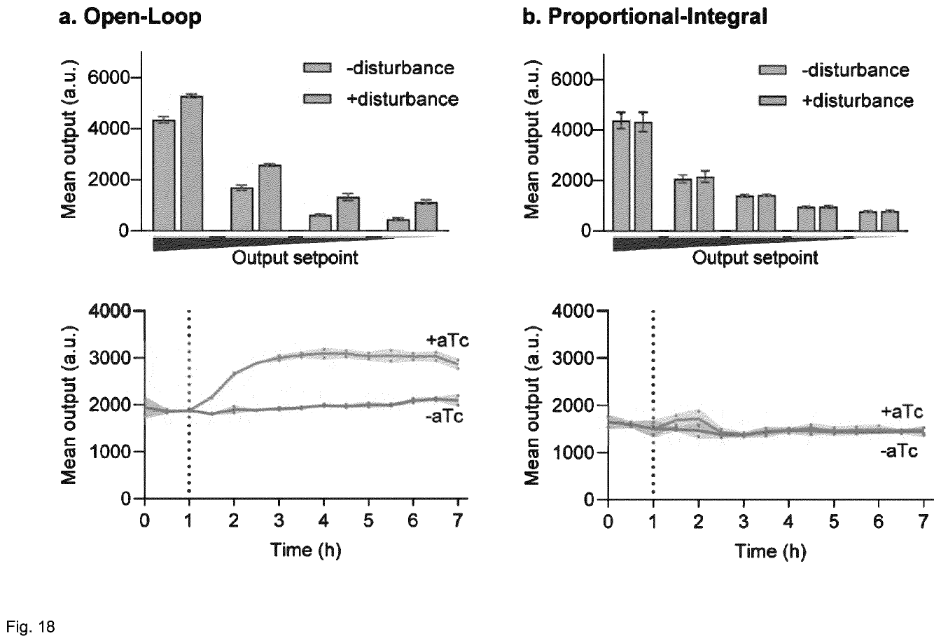

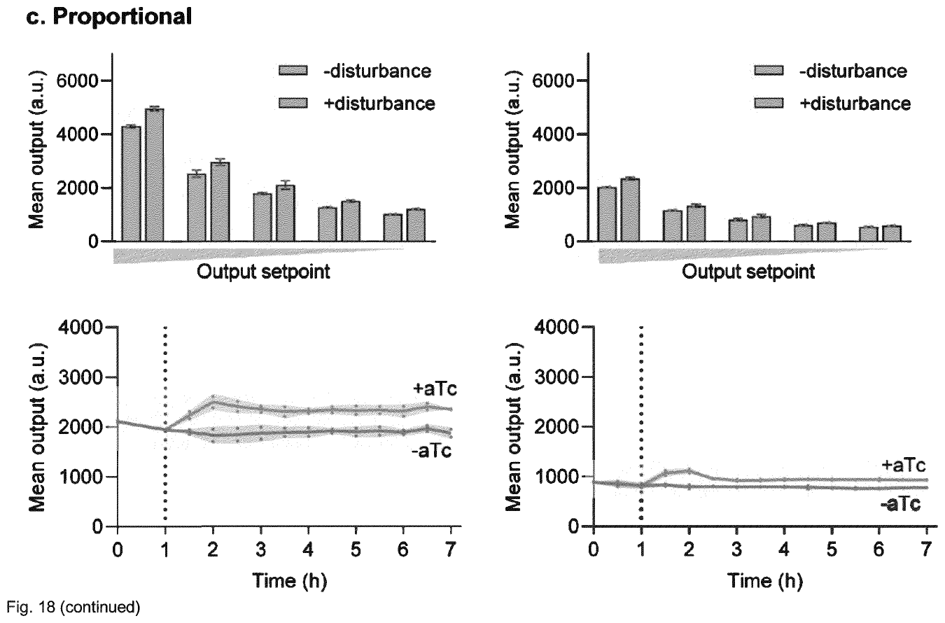

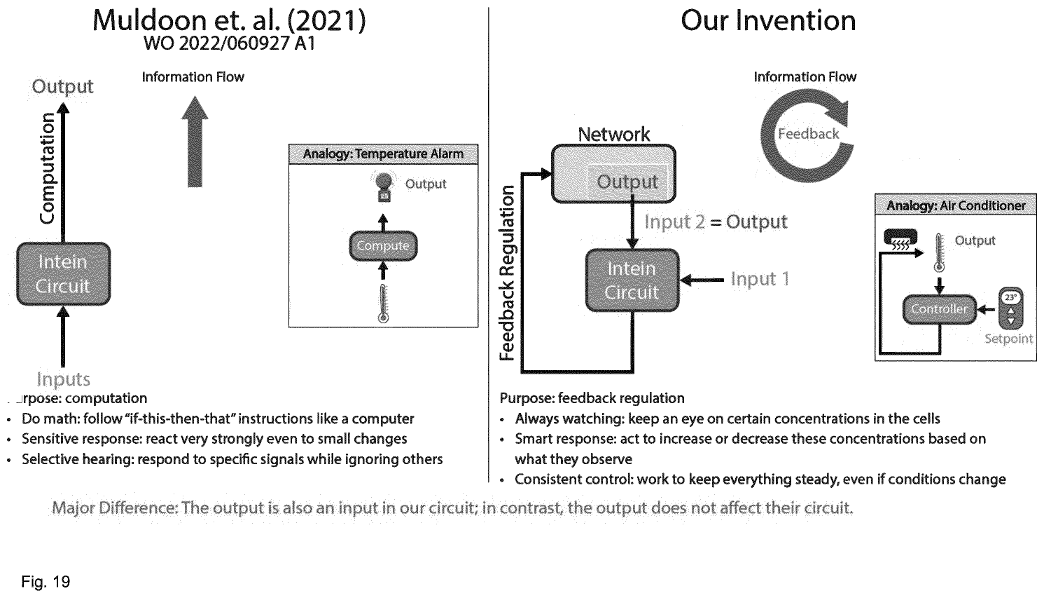

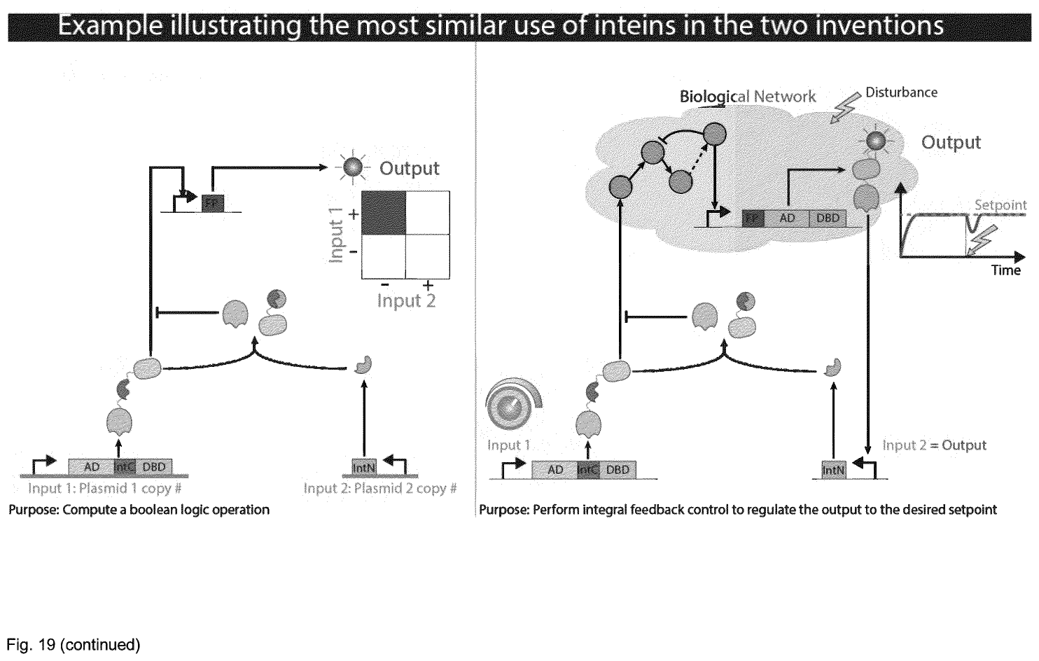

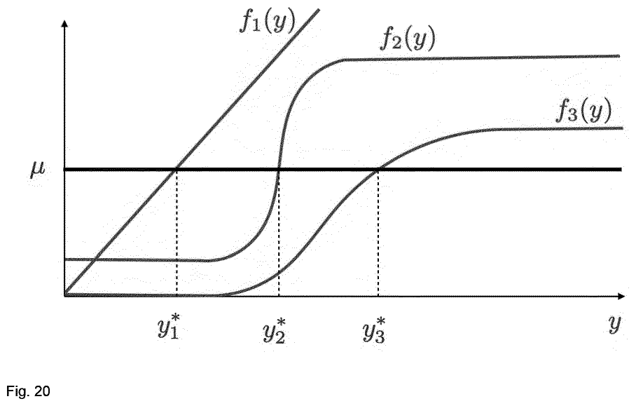

The resulting PI circuit can be tuned using small molecular inducers. First, addition of arabinose increases the affinity of V5-AraC-mScarlet-I for its cognate promoter and thus increases the transcription rate of the tetR::intC. Second, addition of anhydrotetracycline (aTc) decreases the affinity of TetR-IntC for its cognate promoter, leading to decreased repression of V5::araC::mScarlet-I. In a similar way, a proportional controller without integral feedback can be constructed by exchanging intN for intC, which does not interact with tetR::intC, preventing the annihilation reaction from occurring (FIG. 17). For comparison, an open-loop no-feedback control can also be built with and without tetR::intC by replacing the AraC-inducible promoter with a constitutive one, thereby breaking all feedback (FIG. 17). The three circuit types can be directly compared for their ability to achieve robust perfect adaptation by adding a constant amount of aTc to the cultures to act as a disturbance on the output. Output levels at steady-state, measured by flow cytometry and represented as the mean mScarlet-I fluorescence of a population of cells, show that the open-loop circuit at various output setpoints shows an increase in output and failure to achieve robust perfect adaptation, when disturbed with aTc (FIG. 18a, top). Failure to achieve RPA, in this case, is attributed to a lack of feedback as open-loop circuits both with and without TetR-IntC/IntN annihilation reactions were tested. The proportional-integral controller at various output setpoints, on the other hand, shows no change in output when disturbed with aTc (FIG. 18b, top), which also experimentally validates the use of repressor-based controllers in vivo to achieve robust perfect adaptation, an achievement not demonstrated experimentally in the previous examples. The proportional controller, which also uses feedback but lacks the TetR-IntC/IntN annihilation reaction, shows less change in output setpoints as compared to the open-loop but fails to achieve robust perfect adaptation (FIG. 18, top), thus demonstrating that feedback alone is not sufficient for RPA and that the TetR-IntC/IntN annihilation reaction is required.

The same circuits also show that the PI circuit is capable of maintaining an output setpoint over time and that the introduction of an aTc disturbance initially changes the output setpoint but the controller is capable of quickly returning the output to its original setpoint (FIG. 18b, bottom). The open-loop circuit, on the other hand, shows a small drift in setpoint over time and does not reject the aTc disturbance in agreement with the steady-state experiment (FIG. 18a). The proportional controller, like the PI circuit, also maintains the output setpoint over time but does not fully reject the aTc disturbance, in agreement with the steady-state experiment (FIG. 18c).

The intein-based PI controller reported here differs from the integral feedback controller described in D1 in multiple ways. First, the circuit in D1 used a naturally-occurring sequestration pair, sigma/anti-sigma. In contrast, the PI circuit uses inteins to synthetically convert a naturally-occurring repressor, TetR, into a sequestration pair TetR-IntC/IntN. Second, the circuit in D1 uses positive actuation on the output whereas the PI circuit uses negative actuation. As a result, the actuating molecule shifts from Z1 (D1 circuit) to Z2 (PI circuit), resulting in a different topology. Third, The PI circuit is more compact and requires fewer genes than the D1 integral feedback circuit even though it adds an additional layer of proportional feedback to the integral feedback.

### Example 6: Sequences

### Controllers/Co-Controllers:

VP64-IntC(GP41-1)-ZF
(SEQ ID NO: 1)
MPKKKRKVGSGEFDALDDFDLDMLGSDALDDFDLDMLGSDALDDFDLDMLGSDALDDFDLDMLGSG
GGGSGGGGSGGGGSATMMLKKILKIEELDERELIDIEVSGNHLFYANDILTHNSNRGGGGSGGGGS
GGGGSGTARPGERPFQCRICMRNFSRQDRLDRHTRTHTGEKPFQCRICMRNFSQKEHLAGHLRTHT
GEKPFQCRICMRNFSRRDNLNRHLKTHLRGS
VP64-IntC(NrdJ-1)-ZF
(SEQ ID NO: 2)
MPKKKRKVGSGEFDALDDFDLDMLGSDALDDFDLDMLGSDALDDFDLDMLGSDALDDFDLDMLGSG
GGGSGGGGSGGGGSATMGSMEAKTYIGKLKSRKIVSNEDTYDIQTSTHNFFANDILVHNSEIGSNRG
GGGSGGGGSGGGGSGTARPGERPFQCRICMRNFSRQDRLDRHTRTHTGEKPFQCRICMRNFSQK
EHLAGHLRTHTGEKPFQCRICMRNFSRRDNLNRHLKTHLRGS
GAL4(DBD)_IntC(GP41-1)_GAL4(DD)-VPR(212)
(SEQ ID NO: 3)
MKLLSSIEQACDICRLKKLKCSKEKPKCAKCLKNNWECRYSPKTKRSPLTRAHLTEVESRLERLEGSM
LKKILKIEELDERELIDIEVSGNHLFYANDILTHNSGSGSQLFLLIFPREDLDMILKMDSLQDIKALLTGLF
VQDNVNKDAVTDRLASVETDMPLTLRQHRISATSSSEESSNKGQRQLTVSGSSEASGSGRADALDD
FDLDMLGSDALDDFDLDMLGSDALDDFDLDMLGSDALDDFDLDMLINSRSSGSPKKKRKVGSQYL
PDTDDRHRIEEKRKRTYETFKSIMKKSPFSGPTDPRPPPRRIAVPSRSSASVPKPAPQPYPFTSSLST
INYDEFPTMVFPSGQISQASALAPAPPQVLPQAPAPAPAPAMVSALAQAPAPVPVLAPGPPQAVAP
PAPKPTQAGEGTLSEALLQLQFDDEDLGALLGNSTDPAVFTDLASVDNSEFQQLLNQGIPVAPHTT
EPMLMEYPEAITRLVTGAQRPPDPAPAPLGAPGLPNGLLSGDEDFSSIADMDFSALLGSGSGSRDS
REGMFLPKPEAGSAISDVFEGREVCQPKRIRPFHPPGSPWANRPLPASLAPTPTGPVHEPVGSLTP
APVPQPLDPAPAVTPEASHLLEDPDEETSQAVKALREMADTVIPQKEEAAICGQMDLSHPPPRGHL
DELTTTLESMTEDLNLDSPLTPELNEILDTFLNDECLLHAMHISTGLSIFDTSLFRS
tetR_IntC(GP41-1)_VP64
(SEQ ID NO: 4)
MSRLDKSKVINSALELLNEVGIEGLTTRKLAQKLGVEQPTLYWHVKNKRALLDALAIEMLDRHHTHFCP
LEGESWQDFLRNKAKSFRCALLSHRDGAKVHLGTRPTEKQYETLENQLAFLCQQGFSLENALYALSA
VGHFTLGCVLEDQEHQVAKEERETPTTDSMPPLLRQAIELFDHQGAEPAFLFGLELIICGLEKQLKCES
GDSGGGGSMLKKILKIEELDERELIDIEVSGNHLFYANDILTHNSGSGSGRADALDDFDLDMLGSDAL
DDFDLDMLGSDALDDFDLDMLGSDALDDFDLDMLIN
tetR(1-183)_IntC(GP41-1)_tetR(184-212)_VPR
(SEQ ID NO: 5)
MSRLDKSKVINSALELLNEVGIEGLTTRKLAQKLGVEQPTLYWHVKNKRALLDALAIEMLDRHHTHFCP
LEGESWQDFLRNNAKSFRCALLSHRDGAKVHLGTRPTEKQYETLENQLAFLCQQGFSLENALYALSA
VGHFTLGCVLEDQEHQVAKEERETPTTDSMPPLLRQAIELFDHQGAEPGSMLKKILKIEELDERELIDI
EVSGNHLFYANDILTHNSGSGSAFLFGLELIICGLEKQLKCESGSPADALDDFDLDMLPADALDDFDLD
MLPADALDDFDLDMLPGQPGSSEASGSGRADALDDFDLDMLGSDALDDFDLDMLGSDALDDFDLD
MLGSDALDDFDLDMLINSRSSGSPKKKRKVGSQYLPDTDDRHRIEEKRKRTYETFKSIMKKSPFSG
PTDPRPPPRRIAVPSRSSASVPKPAPQPYPFTSSLSTINYDEFPTMVFPSGQISQASALAPAPPQVLP
QAPAPAPAPAMVSALAQAPAPVPVLAPGPPQAVAPPAPKPTQAGEGTLSEALLQLQFDDEDLGAL
LGNSTDPAVFTDLASVDNSEFQQLLNQGIPVAPHTTEPMLMEYPEAITRLVTGAQRPPDPAPAPLGA
PGLPNGLLSGDEDFSSIADMDFSALLGSGSGSRDSREGMFLPKPEAGSAISDVFEGREVCQPKRIR
PFHPPGSPWANRPLPASLAPTPTGPVHEPVGSLTPAPVPQPLDPAPAVTPEASHLLEDPDEETSQA
VKALREMADTVIPQKEEAAICGQMDLSHPPPRGHLDELTTTLESMTEDLNLDSPLTPELNEILDTFLN
DECLLHAMHISTGLSIFDTSLFRS
TetR-dead IntN
(SEQ ID NO: 6)
MSRLDKSKVINSALELLNEVGIEGLTTRKLAQKLGVEQPTLYWHVKNKRALLDALAIEMLDRHHTHFCP
LEGESWQDFLRNNAKSFRCALLSHRDGAKVHLGTRPTEKQYETLENQLAFLCQQGFSLENALYALSA
VGHFTLGCVLEDQEHQVAKEERETPTTDSMPPLLRQAIELFDHQGAEPAFLFGLELIICGLEKQLKCES
GSGGGGSGGGGTGGGGTGTGYNEVLNVFPKSKKKSYKITLEDGKEIICSEEHLFPTQTGEMNISGG
LKEGMCLYVKEGST
IntC-VPR
(SEQ ID NO: 7)
MMLKKILKIEELDERELIDIEVSGNHLFYANDILTHNSGSGSSEASGSGRADALDDFDLDMLGSDALD
DFDLDMLGSDALDDFDLDMLGSDALDDFDLDMLINSRSSGSPKKKRKVGSQYLPDTDDRHRIEEKR
KRTYETFKSIMKKSPFSGPTDPRPPPRRIAVPSRSSASVPKPAPQPYPFTSSLSTINYDEFPTMVFPS
GQISQASALAPAPPQVLPQAPAPAPAPAMVSALAQAPAPVPVLAPGPPQAVAPPAPKPTQAGEGT
LSEALLQLQFDDEDLGALLGNSTDPAVFTDLASVDNSEFQQLLNQGIPVAPHTTEPMLMEYPEAITR
LVTGAQRPPDPAPAPLGAPGLPNGLLSGDEDFSSIADMDFSALLGSGSGSRDSREGMFLPKPEAG
SAISDVFEGREVCQPKRIRPFHPPGSPWANRPLPASLAPTPTGPVHEPVGSLTPAPVPQPLDPAPAV
TPEASHLLEDPDEETSQAVKALREMADTVIPQKEEAAICGQMDLSHPPPRGHLDELTTTLESMTEDL
NLDSPLTPELNEILDTFLNDECLLHAMHISTGLSIFDTSLFRSTSK
Anticontrollers/Open-loop controls + Proxy-Reporter:
IntN(Gp41-1)_p2A-t2A_moxVenus
(SEQ ID NO: 8)
MGGGGSGGGGTGGGGTGTRSGYCLDLKTQVQTPQGMKEISNIQVGDLVLSNTGYNEVLNVFPKSK
KKSYKITLEDGKEIICSEEHLFPTQTGEMNISGGLKEGMCLYVKEGSGSGATNFSLLKQAGDVEENPG
PGSGEGRGSLLTCGDVEENPGPIDGGSGGSATMVSKGEELFTGVVPILVELDGDVNGHKFSVSGEG
EGDATYGKLTLKLICTTGKLPVPWPTLVTTLGYGLQCFARYPDHMKQHDFFKSAMPEGYVQERTIFFK
DDGNYKTRAEVKFDGDTLVNRIELKGIDFKEDGNILGHKLEYNYNSHNVYITADKQKNGIKANFKIRHNI
EDGGVQLADHYQQNTPIGDGPVLLPDNHYLSYQSKLSKDPNEKRDHMVLLEFVTAAGITLGMDELYK
GSGSGS
IntC(Gp41-1)_p2A-t2A_moxVenus
(SEQ ID NO: 9)
MMLKKILKIEELDERELIDIEVSGNHLFYANDILTHNSGSGSGSGATNFSLLKQAGDVEENPGPGSGE
GRGSLLTCGDVEENPGPIDGGSGGSATMVSKGEELFTGVVPILVELDGDVNGHKFSVSGEGEGDAT
YGKLTLKLICTTGKLPVPWPTLVTTLGYGLQCFARYPDHMKQHDFFKSAMPEGYVQERTIFFKDDGNY
KTRAEVKFDGDTLVNRIELKGIDFKEDGNILGHKLEYNYNSHNVYITADKQKNGIKANFKIRHNIEDGGV
QLADHYQQNTPIGDGPVLLPDNHYLSYQSKLSKDPNEKRDHMVLLEFVTAAGITLGMDELYKGSGSG
S
IntN(NrdJ-1)_p2A-t2A_moxVenus
(SEQ ID NO: 10)
MNPCCLVGSSEIITRNYGKTTIKEVVEIFDNDKNIQVLAFNTHTDNIEWAPIKAAQLTRPNAELVELEIDT
LHGVKTIRCTPDHPVYTKNRGYVRADELTDDDELVVAIGSGSGATNFSLLKQAGDVEENPGPGSGEG
RGSLLTCGDVEENPGPIDGGSGGSATMVSKGEELFTGVVPILVELDGDVNGHKFSVSGEGEGDATY
GKLTLKLICTTGKLPVPWPTLVTTLGYGLQCFARYPDHMKQHDFFKSAMPEGYVQERTIFFKDDGNYK
TRAEVKFDGDTLVNRIELKGIDFKEDGNILGHKLEYNYNSHNVYITADKQKNGIKANFKIRHNIEDGGVQ
LADHYQQNTPIGDGPVLLPDNHYLSYQSKLSKDPNEKRDHMVLLEFVTAAGITLGMDELYKGSGSGS
IntC(NrdJ-1)_p2A-t2A_moxVenus
(SEQ ID NO: 11)
MEAKTYIGKLKSRKIVSNEDTYDIQTSTHNFFANDILVHNSEIGSGSGATNFSLLKQAGDVEENPGPGS
GEGRGSLLTCGDVEENPGPIDGGSGGSATMVSKGEELFTGVVPILVELDGDVNGHKFSVSGEGEGD
ATYGKLTLKLICTTGKLPVPWPTLVTTLGYGLQCFARYPDHMKQHDFFKSAMPEGYVQERTIFFKDDG
NYKTRAEVKFDGDTLVNRIELKGIDFKEDGNILGHKLEYNYNSHNVYITADKQKNGIKANFKIRHNIEDG
GVQLADHYQQNTPIGDGPVLLPDNHYLSYQSKLSKDPNEKRDHMVLLEFVTAAGITLGMDELYKGSG
SGS

### Example 7: Materials and Methods

### Data Labelling

Illustration of the experimental setup for closed loop characterization is shown in FIG. 3. The controllers were tested in a two-plasmid system. Plasmid 1 encodes the activator with an IntC incorporated under the control of a constitutive output. Plasmid 2 encodes either IntC (in the open loop setting) or IntN (in the closed loop setting), which is fused to the fluorophore mVenus via a P2A-T2A linker. The P2A-T2A leads to ribosome skipping, which leads to the translation of two separate proteins (IntN and mVenus) in a fixed ration from a common mRNA. The mVenus is used as a proxy reporter for the its own mRNA which the controlled output. The gene expression on Plasmid 2 is regulated by the activators expressed from Plasmid 1. In the open loop there is no interaction between the two IntC as they are unable to recognize each other and perform the protein splicing reaction. In the closed loop, the pair can heterodimerize and perform the protein splicing reaction that generates further products, which are able to act on the controlled network. The setpoint is regulated by tuning the constitutive production rate, denoted by μ, via the transfected amount of Plasmid 1. The controller “senses” the abundance of the controlled output via the translation of the mRNA encoded in Plasmid 2, whose rate is denoted by θ. Furthermore, Plasmid 2 is used for disturbances as its concentration does not alter the setpoint which is equal to μ/θ. The amount of transfected Plasmid 2 is fixed to a certain value for all setpoints and the output is regarded as the reference. The same experiment is repeated for all setpoints with a higher amount of Plasmid 2 and is regarded as the disturbed output.

A simplified schematic of the controller topology can be seen on the left panel of FIGS. 4-7 together with a selected subset of the controller species. A green (resp. red) background indicates upregulation (resp. downregulation) of the output concentration; whereas, a grey background indicates no impact on the output concentration. The middle panel shows the normalized fluorescent data of the proxy reporter. The disturbed and reference triplicate measurements were normalized to the mean fluorescence of the reference data for every setpoint. The X-axis is grouped to correspond to the closed- and open-loop circuits, and shows the amount of Plasmid 1 transfected within every well. The y-axis shows the normalized fluorescent output. The red horizontal line gives the normalized output averaged over all setpoints. The labeling above the lines indicates the average error in % compared to the reference data. The right panel shows the non-normalized data over a selected subset of setpoint that match in fluorescence between the open- and the closed-loop circuits.

### Plasmid Construction

All plasmids were generated with a mammalian adaptation of the modular cloning (MoClo) yeast toolkit standard. All individual parts were generated by PCR amplification (Phusion Flash High-Fidelity PCR Master Mix; Thermo Scientific) or synthesized with Twist Bioscience. The parts were then assembled with golden gate assembly. All enzymes for plasmid construction were obtained from New England Biolabs (NEB). Constructs were chemically transformed into E. coli Top10 strains.

### Cell Culture

All experiments were performed with HEK293T cells (ATCC, strain number CRL-3216). The cells were cultured in Dulbecco's modified Eagle's medium (DMEM; Gibco) supplemented with 10% FBS (Sigma-Aldrich), 1× GlutaMAX (Gibco), 1 mm Sodium Pyruvate (Gibco), penicillin (100 U/μL), and streptomycin (100 μg/mL) (Gibco) at 37° C. with 5% CO2. The cell culture was passaged into a fresh T25 flask (Axon Lab) every 2 to 3 days. Upon detachment some part of the cell suspension was used for the transfection.

### Transfection

All plasmids were isolated using ZR Plasmid Miniprep-Classic (Zymo Research). The plasmids were introduced to the HEK293T cells via suspension transfection. A transfection solution in Opti-MEM I (Gibco) was prepared using Polyethylenimine (PEI) “MAX” (MW 40000; Polysciences, Inc.) at a 1:3 (μg DNA to μg PEI) ratio while the culture was detached with Trypsin-EDTA (Gibco). The cell density was assessed with the automated cell counter Countess II FL (Invitrogen). 100 μL of culture with 26000 cells was transferred in each well of the plate Nunc Edge 96-well plate (Thermo Scientific). The transfection mixture was added to the cells once it has incubated for approximately 30 min.

### Flow Cytometry

The cells were detached approximately 48 hours after transfection on the Eppendorf ThermoMixer C at 25° C. at 700 rpm with 53 μL Accutase solution (Sigma-Aldrich) per well for 20 min. The fluorescence data was collected on the Beckman Coulter CytoFLEX S flow cytometer with the 488 nm excitation with a 525/40+OD1 bandpass filter and the 638 nm excitation with a 660/10 bandpass filter. All data was processed with the CytExpert 2.3 software.

### Stable Cell Line Generation for Example 3

Stable HEK293T cell lines were produced using the PiggyBac transposase system. Specifically, cells were co-transfected with a PiggyBac transposon vector carrying the synthetic circuit and a PiggyBac transposase expression vector. In the case of controller cells, two transposon vectors were simultaneously integrated. A poly-transfection method was used, resulting in a random copy number of the two synthetic circuits in the cells. Each transposon vector also carried an antibiotic resistance marker for selection and a fluorescence reporter gene for cell type identification during co-culture experiments or for ratio screening between synthetic circuits. Transfections were conducted using polyethylenimine (PEI) “MAX” (MW 40000; Polysciences, Inc.). The ratio between micrograms of DNA to micrograms of PEI was 1:3, with a total of 1800 ng of plasmid DNA used per well of a 6-well plate. Each well, pre-seeded with 150,000 cells 24 hours before treatment. Antibiotics were introduced into the culture medium 72 hours post-transfection to select for successfully transfected cells. The type of antibiotics used was based on the resistance markers present on the transposon vector. To further confirm the successful transgene integration and expression, polyclonal cell lines were subjected to fluorescence-activated cell sorting (FACS) analysis based on the fluorescence reporter gene expression. Cells were washed and resuspended in PBS prior to sorting with a BD FACSAria™ Fusion Cell Sorter (BD Biosciences). Single cells exhibiting the fluorescent reporter gene were isolated and expanded to generate monoclonal cell lines. After expansion, cells were adapted to suspension culture following the guidelines of the SFM II medium (Thermo Fisher Scientific) for future experiments.

### Co-Culture Time Course Assay

To set up the co-culture experiments, three types of cells, namely controller cells, reporter cells, and TNF-producing cells, were initially seeded in a 24-well plate one day before the experiment. The seeding density was specified at 300,000 cells/mL, with a total volume of 500 μL per well. The ratio among the three cell types was configured to 80% controller cells, 18% reporter cells, and 2% TNF-producing cells. Throughout the experimental period, fluorescence measurements were carried out daily using a Beckman Coulter CytoFLEX S flow cytometer. Concurrently, fresh medium, containing varying concentrations of DOX, was introduced daily to induce disturbance. The total volume per well was consistently sustained at 500 μL, implementing a daily dilution rate of 60%. For data analysis, a custom pipeline developed in the R programming language was employed. The measured events were automatically gated and compensated, thereby facilitating subsequent plotting and in-depth analysis.

### Stable Cell Line Generation for Example 4

Stable HEK293T cell lines were produced using the PiggyBac transposase system. Specifically, cells were co-transfected with a PiggyBac transposon vector carrying the synthetic circuit and a PiggyBac transposase expression vector. To generate the controller cells, two transposon vectors were simultaneously integrated. A poly-transfection method was used, resulting in a random copy number of the two synthetic circuits in the cells. Each transposon vector also carried an antibiotic resistance marker for selection Transfections were conducted using polyethylenimine (PEI) “MAX” (MW 40000; Polysciences, Inc.). The ratio between micrograms of DNA to micrograms of PEI was 1:3, with a total of 1200 ng of plasmid DNA used for each well of a 6-well plate. Each well was pre-seeded with 300,000 cells 24 hours before treatment. Antibiotics were introduced into the culture medium 72 hours post-transfection to select for successfully transfected cells. The type of antibiotics used was based on the resistance markers present on the transposon vector. To further confirm the successful transgene integration and expression, polyclonal cell lines were subjected to fluorescence-activated cell sorting (FACS) analysis based on the fluorescence reporter gene expression. Cells were washed and resuspended in PBS prior to sorting with a BD FACSAria™ Fusion Cell Sorter (BD Biosciences). Clones that survived antibiotic selection were screened for light induction, using an in-house developed programmable light illumination array and flow cytometry. Functional clones were used for the Cyberloop experiments and live cell imaging.

### Live Cell Imaging

Before Cyberloop experiments, 15,000 cells were seeded per well of a 24-well plate, pre-coated with 0.2% gelatin (VisiPlate, PerkinElmer). Each illumination pulse was performed using the 4× objective of the microscope (duration: 7.5 sec, sampling period: 13 min).

### Mathematical Modeling

The network simulations are implemented in the deterministic setting using the MATLAB (MathWorks) environment. The mathematical whole-body type 1 diabetes model describing the dynamics of the glucose-insulin system is adopted from (Dalla Man et al. 2007. IEEE Transactions on Bio-Medical Engineering 54 (10): 1740-49.). For the open-loop simulations (red lines in FIG. 2d), kₚ₁∈[2.4, 3], Vₘₓ∈[0.024, 0.071], and kₑ₁∈[0.0003, 0.0008] in the controlled network are varied. Changes of kₚ₁ depict alterations in endogenous glucose production [e.g., in various catabolic or stress states], and Vₘₓ is used to simulate variations in the insulin-dependent glucose utilization in the peripheral tissues (e.g., by physiological or pathological changes in GLUT4 translocation), while kₑ₁ is the glomerular filtration rate.

### Imaging & Light Delivery System

Images were taken under a Nikon Ti2-E inverted microscope (Nikon Instruments), equipped with a 4× (MRD70040) objective, acquired from Nikon AG, Egg, Switzerland. One CMOS camera ORCA-Flash4.0 LT PLUS (Hamamatsu Photonic, Solothurn, Switzerland) was also integrated with the microscope. The following imaging set-ups were used in the microscope. Brightfield imaging, default Nikon DiaLamp with diffuser and green interference filter placed in the light path; fluorescence imaging, Spectra Ill Light Engine fluorescence excitation light source (Lumencor, Beaverton, USA); Cy3 (mScarlet-1) imaging: 561/40 nm LED line, HC 573 beam splitter, 600/32 nm emission filter; Cy5 (miRFP670) imaging, 640/30 nm LED line, HC-BS660 beam splitter, 692/40 nm emission filter. All filters and beam splitters were acquired from AHF Analysetechnik AG, Tubingen, Germany.

### Growth Conditions:

Escherichia coli cells were grown in M9 medium supplemented with 0.2% casamino acids, 0.4% glucose, 0.001% thiamine, 0.00006% ferric citrate, 0.1 mM calcium chloride, 1 mM magnesium sulfate, and 20 μg/mL uracil (Sigma-Aldrich Chemie GmbH), and incubated in an environmental shaker (New Brunswick) at 37° C. with shaking at 230 rpm. Antibiotics (Sigma-Aldrich Chemie GmbH) were used at the following concentrations: carbenicillin (carb), 100 μg/mL; spectinomycin (spec), 100 μg/mL; chloramphenicol (cam), 34 μg/mL.

E. coli Host Strain:

Host strain SKA360 (MG1655 ΔaraCBAD ΔlacIZYA ΔaraE ΔaraFGH attB::acYA177C ΔrhaSRT ΔrhaBADM) is a precursor strain to SKA703 constructed as previously described in (Aoki et al. 2019. Nature 570 (7762): 533-37.).

E. coli Plasmids:

All plasmids were constructed from a custom-made library of parts with optimized overhangs (Potapov et al. 2018. ACS Synthetic Biology 7 (11): 2665-74.) using standard Golden-Gate assembly methods and modular cloning (MoClo) (Marillonnet et al. 2020. Current Protocols in Molecular Biology/Edited by Frederick M. Ausubel 130 (1): e115.) with restriction enzymes BsaI and BbsI (New England Biolabs). Circuit modules were split between three different plasmids.

Output plasmid consists of a V5::araC::mScarlet-I fusion(Aoki et al. 2019. Nature 570 (7762): 533-37.), (Bindels et al. 2017. Nature Methods 14 (1): 53-56.) under the control of a P_{LtetO-1} promoter (Lutz and Bujard. 1997. Nucleic Acids Research 25 (6): 1203-10.) and weak Bba_b0033 ribosomal binding site (RBS) from the Registry of Standard Biological Parts on a high copy plasmid with ColE1 origin of replication and beta-lactamase (carb^{R}) gene.

The TetR-IntC plasmids consist of a tetR₁₈₄::intC(GP41-1) fusion (Anastassov et al. 2023. Nature Communications 14 (1): 1337.) under the control of either a modified Pₐᵣₐ promoter (Aoki et al. 2019. Nature 570 (7762): 533-37.) and weak B0033 RBS or AraJ-B0033m ribozyme/RBS (for the proportional and proportional-integral circuits) (Lou et al. 2012. Nature Biotechnology 30 (11): 1137-42.) or a Bba_J23111 or Bba_J23119 constitutive promoter from the Registry of Standard Biological Parts and weak B0033 RBS on a low copy plasmid with pSC101 origin of replication and chloramphenicol-acyltransferase (cam^{R}) gene.

The IntN/IntC plasmid contains either intN(GP41-1) or intC(GP41-1) (Carvajal-Vallejos et al. 2012. The Journal of Biological Chemistry 287 (34): 28686-96.) under a J23119 constitutive promoter and weak B0033 RBS on a medium copy plasmid with p15A origin of replication and aminoglycoside adenylyltransferase (spec^{R}) gene.

Plasmids were transformed into E. coli host strain SKA360 as previously described (Chung et al. 1989. Proceedings of the National Academy of Sciences of the United States of America 86 (7): 2172-75.).

E. coli Steady-State Experiments:

200 μl aliquots of M9 medium in 96-well flat-bottom plates (Greiner) with appropriate antibiotics were inoculated with the circuit strains from glycerol freeze stocks. The plates were covered with BreathSeal film (Greiner) and a plastic lid (Greiner) and were incubated overnight at 37° C. with shaking to stationary phase. In the morning, cultures were diluted 1:1,200,000 in fresh 200 μl aliquots of M9 medium in 96-well flat-bottom plates containing arabinose (Sigma-Aldrich) at final concentrations of 0.2%, 0.35%, 0.5%, 0.75%, and 1% with or without 0.5 ng/mL anhydrotetracycline (aTc, Chemie Brunschwig). Plates were covered with BreathSeal film and plastic lids and incubated for six hours at 37° C. with shaking. After six hours of shaking, all cultures are in exponential phase (optical density at 600 nm (OD) less than 0.1). As previously described, cell growth, transcription, and translation are stopped with a rifampicin tetracycline solution and the mScarlet-I was matured for three hours at 37° C. (Baumschlager et al. 2017. ACS Synthetic Biology 6 (11): 2157-67.). mScarlet-I fluorescence was measured on a CytoFlex S flow cytometer (Beckman Coulter) with a 561 nm laser and 610/20 band pass filter; the gain settings were as follows: forward scatter 100, side scatter 100, mScarlet-I 1000. Thresholds of 2,500 FSC-H and 1,000 SSC-H were used for all samples. At least 20,000 events were recorded and cells were manually gated on an FSC-H/SSC-H plot corresponding to the experimentally determined size of the testing strain at logarithmic growth and was kept constant for analysis of all samples using CytExpert Software (Beckman Coulter). The mean fluorescence (ECD-A) for gated cells was calculated using CytExpert Software (Beckman Coulter). Data is plotted using Prism 9.4.1.

E. coli Dynamic Experiments:

For this experiment, it is important that the cells are kept in exponential phase to avoid any differences in cell behavior due to growth phase. A 3 mL aliquot of M9 medium containing appropriate antibiotics and 0.5% arabinose was inoculated with cells from glycerol freeze stocks at a low OD so that after approximately 10 hours of incubation overnight at 37° C. and 230 rpms, cultures are at an OD between 0.01 and 0.03. The exponential phase culture was then used to start pseudo-time course experiments. Briefly, the time courses are split into two phases. The first phase is one hour of growth in 0.5% arabinose to ensure that the cultures are at steady-state and to assess the output level without any disturbance. The second phase is six additional hours of growth in 0.5% arabinose with or without a constant 0.5% aTc disturbance. Cultures for time points 0-1 h are serially diluted and started simultaneously and sampled every 30 minutes. After 1 h of growth, cultures for time points 1.5-7 h (with and without aTc) are serially diluted and started simultaneously and sampled every 30 minutes. This dilution and sampling strategy ensures that every time point collected contains approximately the same amount of exponentially-growing cells (OD less than 0.1). After collecting all the time points, mScarlet-I is matured for all the samples at the same time and matured samples are measured at the same time on the flow cytometer.

### CITED PRIOR ART DOCUMENTS

1. C. Kemmer, M. Gitzinger, M. Daoud-El Baba, V. Djonov, J. Stelling, and M. Fussenegger, “Self-sufficient control of urate homeostasis in mice by a synthetic circuit,” Nature biotechnology, vol. 28, no. 4, p. 355, 2010.
2. K. Rössger, G. Charpin-El-Hamri, and M. Fussenegger, “A closed-loop synthetic gene circuit for the treatment of diet-induced obesity in mice,” Nature communications, vol. 4, no. 1, pp. 1-9, 2013.
3. M. Xie, H. Ye, H. Wang, G. Charpin-El Hamri, C. Lormeau, P. Saxena, J. Stelling, and M. Fussenegger, “p-cell-mimetic designer cells provide closed-loop glycemic control,” Science, vol. 354, no. 6317, pp. 1296-1301, 2016.
4. F. Xiao and J. C. Doyle, “Robust perfect adaptation in biomolecular reaction networks,” in 2018 IEEE Conference on Decision and Control (CDC). IEEE, 2018, pp. 4345-4352.
5. M. H. Khammash, “Perfect adaptation in biology,” Cell Systems, vol. 12, no. 6, pp. 509-521, 2021.
6. B. A. Francis and W. M. Wonham, “The internal model principle of control theory,” Automatica, vol. 12, no. 5, pp. 457-465, 1976.
7. C. Briat, A. Gupta, and M. Khammash, “Antithetic integral feedback ensures robust perfect adaptation in noisy biomolecular networks,” Cell Systems, vol. 2, no. 1, pp. 15-26, 2016. [Online]. Available: https://doi.org/10.1016/j.cels.2016.01.004
8. M. Filo, S. Kumar, and M. Khammash, “A hierarchy of biomolecular proportional-integral-derivative feed-back controllers for robust perfect adaptation and dynamic performance,” Nature Communications, vol. 13, no. 1, pp. 1-19, 2022.
9. S. K. Aoki, G. Lillacci, A. Gupta, A. Baumschlager, D. Schweingruber, and M. Khammash, “A universal biomolecular integral feedback controller for robust perfect adaptation,” Nature, p. 1, 2019.
10. T. Frei, C.-H. Chang, M. Filo, A. Arampatzis, and M. Khammash, “Genetically engineered proportional-integral feedback controllers for robust perfect adaptation in mammalian cells,” bioRxiv, 2021. [Online]. Available: https://www.biorxiv.orp/content/early/2021/08/13/2020.12.06412304
11. Briat et al., 2016. Cell Systems 2 (1): 15-26.
12. Kumar et al. 2021. Nature Communications 12 (1): 5651.
13. Wang et al 2012. Nature Methods 9 (3): 266-69.
14. Dalla Man et al. 2007. IEEE Transactions on Bio-Medical Engineering 54 (10): 1740-49.
15. Potapov et al. 2018. ACS Synthetic Biology 7 (11): 2665-74.
16. Marillonnet et al. 2020. Current Protocols in Molecular Biology/Edited by Frederick M. Ausubel 130 (1): e115.
17. Bindels et al. 2017. Nature Methods 14 (1): 53-56.
18. Lutz and Bujard. 1997. Nucleic Acids Research 25 (6): 1203-10.
19. Anastassov et al. 2023. Nature Communications 14 (1): 1337.
20. Lou et al. 2012. Nature Biotechnology 30 (11): 1137-42.
21. Carvajal-Vallejos et al. 2012. The Journal of Biological Chemistry 287 (34): 28686-96.
22. Chung et al. 1989. Proceedings of the National Academy of Sciences of the United States of America 86 (7): 2172-75.
23. Baumschlager et al. 2017. ACS Synthetic Biology 6 (11): 2157-67.

1. A method for constant-level expression of an output, said method comprising the steps:
a. providing a cell or a cell-free system, wherein the cell or the cell-free system is capable of expressing a gene encoding the output;
b. inserting an expression system into said cell or cell-free system, wherein the expression system comprises:
i. a gene encoding a controller, wherein the controller comprises:
I. an optional first effector;
II. a first split-intein; and
III. an optional second effector;
ii. a gene encoding an anti-controller, wherein the anti-controller comprises:
I. an optional third effector;
II. a second split-intein; and
III. an optional fourth effector;
wherein
at least one of the optional effectors is present;
one of the first split-intein and the second split-intein is an N-intein and the other one is a C-intein;
the controller and the anti-controller are capable of undergoing a trans-splicing reaction via the steps:
excising the first split-intein from the controller, and excising the second split-intein from the anti-controller, and
a) if the first split-intein is an N-intein, and the second split-intein is a C-intein:
rejoining
the first effector if present with
the fourth effector if present
yielding a first splice-product;
assembling a protein complex of
the N-intein
bound to the second effector if present
with
the C-intein
bound to the third effector if present
yielding a second splice-product;
b) if the first split-intein is a C-intein, and the second split-intein is an N-intein:
rejoining
the second effector if present with
the third effector if present yielding a first splice-product;
assembling a protein complex of
the N-intein
bound to the fourth effector if present
with
the C-intein
bound to the first effector if present
yielding a second splice-product;
wherein at least the second splice-product is yielded;
the anti-controller is capable of downregulating the controller stoichiometrically via undergoing the trans-splicing reaction;
the second splice-product and the optional first splice-product have a weaker, neutral, or opposite effect on the concentration of the output in comparison to the effect of the controller;
constant-level expression of the output is achieved via a route selected from the group consisting of:
(1)
the controller upregulates the concentration of the output;
the output upregulates the concentration of the anti-controller;
(2)
the controller downregulates the concentration of the output;
the output downregulates the concentration of the anti-controller;
(3)
the controller upregulates the concentration of the output;
the output downregulates the concentration of the controller;
(4)
the controller downregulates the concentration of the output;
the output upregulates the concentration of the controller;
c. exposing said cell or cell-free system to a condition, wherein the expression level of the output is perturbed from a target level to a perturbed level;
d. equilibration of the expression level of the output back to the target level;
wherein the first, second, third, and fourth effector are peptides or polypeptides, and the first and second intein, the controller, and the anti-controller are polypeptides, and the output is a peptide, a polypeptide, or an mRNA.

2. An expression system for constant-level expression of an output, said system comprising:
a. a gene encoding a controller, wherein the controller comprises:
i.) an optional first effector;
ii.) a first split-intein; and
iii.) an optional second effector;
b. a gene encoding an anti-controller, wherein the anti-controller comprises:
i.) an optional third effector;
ii.) a second split-intein; and
iii.) an optional fourth effector;
wherein
at least one of the optional effectors is present;
one of the first split-intein and the second split-intein is an N-intein and the other one is a C-intein;
the controller and the anti-controller are capable of undergoing a trans-splicing reaction via the steps:
excising the first split-intein from the controller, and excising the second split-intein from the anti-controller, and
a) if the first split-intein is an N-intein, and the second split-intein is a C-intein:
rejoining
the first effector if present with
the fourth effector if present
yielding a first splice-product;
assembling a protein complex of
the N-intein
bound to the second effector if present
with
the C-intein
bound to the third effector if
present
yielding a second splice-product;
b) if the first split-intein is a C-intein, and the second split-intein is an N-intein:
rejoining
the second effector if present with
the third effector if present
yielding a first splice-product;
assembling a protein complex of
the N-intein
bound to the fourth effector if present
with
the C-intein
bound to the first effector if present
yielding a second splice-product;
wherein at least the second splice-product is yielded;
the anti-controller is capable of downregulating the controller stoichiometrically via undergoing the trans-splicing reaction;
the second splice-product and the optional first splice-product have a weaker, neutral, or opposite effect on the concentration of the output in comparison to the effect of the controller;
a route via which constant-level expression of the output is achieved is selected from the group consisting of:
(1)
the controller is capable of upregulating the concentration of the output;
the output is capable of upregulating the concentration of the anti-controller;
(2)
the controller is capable of downregulating the concentration of the output;
the output is capable of downregulating the concentration of the anti-controller;
(3)
the controller is capable of upregulating the concentration of the output;
the output is capable of downregulating the concentration of the controller;
(4)
the controller is capable of downregulating the concentration of the output;
the output is capable of upregulating the concentration of the controller,
wherein the first, second, third, and fourth effector are peptides or polypeptides, and the first and second intein, the controller, and the anti-controller are polypeptides, and the output is a peptide, a polypeptide, or an mRNA.

3. The method according to claim 1, wherein the controller comprises a further first intein and/or the anti-controller comprises a further second intein and the trans-splicing reaction is executed multiple times finally yielding the second splice-product and optionally the first splice-product.

4. The method according to claim 1 wherein for case (1) that
the controller is capable of upregulating the concentration of the output;
the output is capable of upregulating the concentration of the anti-controller;
additionally, the anti-controller is capable of downregulating the concentration of the output.

5. The method according to claim 1, wherein for case (2) that
the controller is capable of downregulating the concentration of the output;
the output is capable of downregulating the concentration of the anti-controller;
additionally, the anti-controller is capable of upregulating the concentration of the output.

6. The method according to claim 1, wherein for case (3) that
the controller is capable of upregulating the concentration of the output;
the output is capable of downregulating the concentration of the controller;
additionally, the anti-controller is capable of downregulating the concentration of the output.

7. The method according to claim 1, wherein for case (4) that
the controller is capable of downregulating the concentration of the output;
the output is capable of upregulating the concentration of the controller;
additionally, the anti-controller is capable of upregulating the concentration of the output.

8. The method according to claim 1, wherein the first intein is inserted into the controller in a loop region of the first and second effector and/or the second intein is inserted into the anti-controller in a loop region of the third and fourth effector.

9. The method according to claim 1, wherein
the first effector, and/or
the second effector, and/or
the third effector, and/or
the fourth effector
comprise a domain of a transcription factor.

10. The method according to claim 9, wherein
the controller is a non-dimerizing transcription factor (TF) modified by insertion of a C-intein;
the first effector is a DNA-binding domain of the TF;
the second effector is an activation domain of the TF;
the anti-controller is an N-intein;
the third and the fourth effectors are not present.

11. The method according to claim 9, wherein
the controller is a dimerizing transcription factor (TF) modified by insertion of a C-intein;
the first effector consists of
a DNA-binding domain of the TF;
an N-terminal part of a dimerization domain of the TF;
the second effector consists of
a C-terminal part of a dimerization domain of the TF;
an activation domain of the TF;
the anti-controller is an N-intein;
the third and the fourth effectors are not present.

12. The method according to claim 9, wherein
the controller is a dimerizing transcription factor (TF) modified by insertion of a C-intein;
the first effector consists of
a DNA-binding domain of the TF;
a dimerization domain of the TF;
the second effector is an activation domain of the TF;
the anti-controller is an N-intein;
the third and the fourth effectors are not present.

13. The method according to claim 9, wherein
the controller is a dimerizing transcription factor (TF) modified by insertion of a C-intein;
the first effector is a DNA-binding domain of the TF;
the second effector consists of
a dimerization domain of the TF;
an activation domain of the TF;
the anti-controller is an N-intein;
the third and the fourth effectors are not present.

14. The method according to claim 9, wherein
the controller is an activation domain modified by fusion of a C-intein;
the second effector is an activation domain;
the first effector is not present;
the anti-controller is an N-intein;
the third and the fourth effectors are not present;
the expression system additionally comprises a co-controller, wherein the co-controller comprises
a DNA-binding domain of the TF;
a dimerization domain of the TF;
an inactive N-intein.

15. The method according to claim 1, wherein the output is a cytokine.

16. The method according to claim 15, wherein
the first effector, and/or
the second effector, and/or
the third effector, and/or
the fourth effector
comprise a domain of an inhibitor of said cytokine, particularly wherein the inhibitor is an antibody.

17. The method according to claim 1, wherein
for case (1): the output is capable of regulating the concentration of the anti-controller proportionally; or
for case (2): the output is capable of regulating the concentration of the anti-controller anti-proportionally; or
for case (3): the output is capable of regulating the concentration of the controller anti-proportionally; or
for case (4): the output is capable of regulating the concentration of the controller proportionally.

18. The method according to claim 17, wherein
for case (1) or (2): the target expression level of the output is tunable via adjusting a production rate of the controller and/or via adjusting a ratio of a production rate of the anti-controller to an output expression level; or
for case (3) or (4): the target expression level of the output is tunable via adjusting a production rate of the anti-controller and/or via adjusting a ratio of a production rate of the controller to an output expression level.

19. The method according to claim 17, wherein
for case (1) or (2): the target expression level of the output is tunable via adjusting a production rate of the controller and/or via adjusting a production rate of the anti-controller, wherein the production rate of the anti-controller is a function of the output expression level; or
for case (3) or (4): the target expression level of the output is tunable via adjusting a production rate of the anti-controller and/or via adjusting a production rate of the controller, wherein the production rate of the controller is a function of the output expression level.

20. A cell comprising the expression system according to claim 2.
